SEQUENCE LISTING (7 sequences)

SEQ ID NO: 1 - protein, 158 aa, synthetic construct
MHHHHHHGSGGSAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASASGSTSAHIVMVDAYKPTK

SEQ ID NO: 2 - protein, 450 aa, synthetic construct
MHHHHHHGSGGSDSATHIKFSKRDEDGKELAGATMELRDSSGKTISTWISDGQVKDFYLYPGKYTFVETAAPDGYEVATAITFTVNEQGQVTVNGKATKGDAHIASYRDPMGAGAGAGPEGAGAGAGPEGAGAGAGPEGAGAGAGPEGAGAGAGPEGAGAGAGPEGAGAGAGPEGAGAGAGPEGAGAGAGPEGAGAGAGPEGARMPTSYRDPMGAGAGAGPEGAGAGAGPEGAGAGAGPEGAGAGAGPEGAGAGAGPEGAGAGAGPEGAGAGAGPEGAGAGAGPEGAGAGAGPEGAGAGAGPEGARMPTSYRDPMGAGAGAGPEGAGAGAGPEGAGAGAGPEGAGAGAGPEGARMPEFDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 3 - protein, 461 aa, synthetic construct
MHHHHHHGSGGSDSATHIKFSKRDEDGKELAGATMELRDSSGKTISTWISDGQVKDFYLYPGKYTFVETAAPDGYEVATAITFTVNEQGQVTVNGKATKGDAHIASGSGTDLGKKLLEAARAGQDDEVRILMANGADVNAKDKDGYTPLHLAAREGHLEIVEVLLKAGADVNAKDKDGYTPLHLAAREGHLEIVEVLLKAGADVNAKDKDGYTPLHLAAREGHLEIVEVLLKAGADVNAKDKDGYTPLHLAAREGHLEIVEVLLKAGADVNAKDKDGYTPLHLAAREGHLEIVEVLLKAGADVNAKDKDGYTPLHLAAREGHLEIVEVLLKAGADVNAQDKFGKTAFDISIDNGNEDLAEILQTSGSEFDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 4 - protein, 360 aa, synthetic construct
MGSGSGSAPQMLRELQETNAALQDVRELLRQQVKEITFLKNTVMESDASGASYRDPMGAGAGAGPEGAGAGAGPEGAGAGAGPEGAGAGAGPEGAGAGAGPEGAGAGAGPEGAGAGAGPEGAGAGAGPEGAGAGAGPEGAGAGAGPEGARMPTSYRDPMGAGAGAGPEGAGAGAGPEGAGAGAGPEGAGAGAGPEGAGAGAGPEGAGAGAGPEGAGAGAGPEGAGAGAGPEGAGAGAGPEGAGAGAGPEGARMPTSYRDPMGAGAGAGPEGAGAGAGPEGAGAGAGPEGAGAGAGPEGARMPEFAPQMLRELQETNAALQDVRELLRQQVKEITFLKNTVMESDASGKLAAALEHHHHHH

SEQ ID NO: 5 - protein, 9 aa, synthetic construct
AGAGAGPEG

SEQ ID NO: 6 - protein, 250 aa, synthetic construct
YRDPMGAGAGAGPEGAGAGAGPEGAGAGAGPEGAGAGAGPEGAGAGAGPEGAGAGAGPEGAGAGAGPEGAGAGAGPEGAGAGAGPEGAGAGAGPEGARMPTSYRDPMGAGAGAGPEGAGAGAGPEGAGAGAGPEGAGAGAGPEGAGAGAGPEGAGAGAGPEGAGAGAGPEGAGAGAGPEGAGAGAGPEGAGAGAGPEGARMPTSYRDPMGAGAGAGPEGAGAGAGPEGAGAGAGPEGAGAGAGPEGARMP

SEQ ID NO: 7 - protein, 253 aa, synthetic construct
DLGKKLLEAARAGQDDEVRILMANGADVNAKDKDGYTPLHLAAREGHLEIVEVLLKAGADVNAKDKDGYTPLHLAAREGHLEIVEVLLKAGADVNAKDKDGYTPLHLAAREGHLEIVEVLLKAGADVNAKDKDGYTPLHLAAREGHLEIVEVLLKAGADVNAKDKDGYTPLHLAAREGHLEIVEVLLKAGADVNAKDKDGYTPLHLAAREGHLEIVEVLLKAGADVNAQDKFGKTAFDISIDNGNEDLAEILQ

(19) United States
(12) Patent Application Publication
(10) Pub. No.: US 2024/0010673 A1
(43) Pub. Date: Jan. 11, 2024

(54) GENETICALLY ENGINEERED RECOMBINANT PROTEINS FOR FUNCTIONAL REGENERATIVE TISSUE SCAFFOLDS

(71) Applicant: ARIZONA BOARD OF REGENTS ON BEHALF OF THE UNIVERSITY OF ARIZONA, Tucson, AZ (US)

(72) Inventors: Minkyu Kim, Tucson, AZ (US); David Storms Knoff, Tucson, AZ (US); Samuel Kim, Tucson, AZ (US)

(21) Appl. No.: 18/365,648

(22) Filed: Aug. 4, 2023

Related U.S. Application Data
(63) Continuation-in-part of application No. PCT/US22/15468, filed on Feb. 7, 2022.
(60) Provisional application No. 63/146,471, filed on Feb. 5, 2021.

(51) Int. Cl. C07K 1/107

(52) U.S. Cl. CPC C07K 1/1077

(57) ABSTRACT

Protein complexes are profound in various biological processes, and their ability to self-associate offers exciting opportunities for innovative biomaterials. However, the weak physical interactions within protein complexes result in limited mechanical stability and fast erosion rates, posing a significant challenge. Thus, a SAv-based hydrogel where SAv tetramers serve as crosslinking junctions is described herein. These mechanically reinforced SAv tetramers significantly enhanced the shear elasticity and decelerated the bioerosion process of the overall material. This simple approach to modulate the mechanical strength of protein complexes as crosslinking junctions present an attractive prospect for a myriad of biotechnological applications, such as biomimetic scaffolds and soft robotics.

### CROSS-REFERENCES TO RELATED APPLICATIONS

This application is a continuation-in-part and claims benefit of PCT Application No. PCT/US2022/015468 filed Feb. 7, 2022, which claims benefit of U.S. Provisional Application No. 63/146,471 filed Feb. 5, 2021, the specifications of which are incorporated herein in their entirety by reference.

### STATEMENT REGARDING FEDERALLY SPONSORED RESEARCH OR DEVELOPMENT

This invention was made with government support under Grant No. 2143126, awarded by National Science Foundation. The government has certain rights in the invention.

### REFERENCE TO AN ELECTRONIC SEQUENCE LISTING

The contents of the electronic sequence listing UNIA 21_01 PCT-CIP.xml; Size: 9,139 bytes; and Date of Creation: Aug. 1, 2023, is herein incorporated by reference in its entirety.

### FIELD OF THE INVENTION

The present invention features methods and compositions directed towards a recombinant polypeptide biopolymer composition effective as nano springs; in particular, the composition can be utilized as a scaffold for regeneration therapy or for drug delivery.

### BACKGROUND OF THE INVENTION

The global market for tissue engineering and regeneration grew from $6.965 billion in 2017 to $9.246 billion in 2018. Specifically, the cardiovascular tissue engineering and regeneration market grew from $787 million in 2017 to $1.081 billion in 2018. The size and fast growth of these markets are attributed to an aging population, limited tissue and organ transplants, improved molecular biology and genetic engineering technologies, the growth rate of surgical interventions, and the massive prevalence of deadly chronic diseases like cardiovascular disease and cancer.

Responsible for 1 in every 4 deaths in the United States, cardiovascular disease is the leading cause of death both nationally and worldwide. With current synthetic polymers lacking adequate long-term efficacy in vivo, tissue engineering technologies present great promise for treating diseased cardiovascular tissue. While many synthetic polymers have been used to develop scaffolds for tissue implants, the limitations in biocompatibility cause debilitating side effects, leading to most surgeons using autograft or allograft natural tissue implants during reconstructive surgeries. Despite the promise, a commercially viable tissue engineered product has yet to reach the market because of the regulatory environment, limited efficacy in vivo, and a lack of cost-effective manufacturing. Two major barriers to creating synthetic polymers for the development of scaffolds for tissue implants.

First, polymer networks are formed by crosslinking junctions connected by strands to form a three-dimensional scaffold. The topology of these polymer networks describes the organization of strands and crosslinking junctions in three-dimensional space. The major challenge of synthesizing polymer-network materials is the lack of organization in the network topology, which consists of many defects that reduce the function and mechanical properties of materials because of ineffective strands and crosslinking junctions. Reducing polymer network defects remains a grand challenge in the field in order to fabricate materials with predictable performance.

The second barrier to designing protein polymer networks that mimic the nanoscale properties of proteins in materials is the lack of a strong and specific cross-linker that overcomes current limitations. The lack of stability, specificity, and temporal control found in current cross-linkers lead to inhomogeneous and disordered polymer networks. While cross-linker research has unveiled promising options, none are able to combine the strength, specificity, and temporal control required to form ordered polymer networks capable of translating protein nanomechanics to macroscale material properties.

The disruptive technology of the present invention introduces a genetically engineered, all-in-one protein design that incorporates key tissue-specific cytoskeleton proteins to produce hydrogel scaffolds capable of mimicking the structure and function of natural tissues in a simplified model. With precise customizability of each protein sequence, these biopolymer tissue grafts have been engineered to achieve biocompatibility and regeneration of natural tissues.

### BRIEF SUMMARY OF THE INVENTION

It is an objective of the present invention to provide compositions and methods that allow for the production of a recombinant polypeptide biopolymer, as specified in the independent claims. Embodiments of the invention are given in the dependent claims. Embodiments of the present invention can be freely combined with each other if they are not mutually exclusive.

Previously the Inventors' successfully demonstrated the ability to reduce topological defects, especially the most debilitating primary loop defects, by incorporating rigid, rod-like proteins in strands between crosslinking junctions. These rigid proteins consisted of a recombinant protein sequence containing 6 ankyrin repeat proteins. Ankyrin, a well-studied protein found in the cytoskeleton of red blood cells (RBCs), is capable of reversibly unfolding and refolding its three-dimensional structure. This property is, in part, responsible for the tremendous reversible deformability of RBCs, allowing them to squeeze through tiny capillaries and arterioles less than half of their diameter before snapping back to their original length after returning to larger blood vessels. The single molecule properties of ankyrin repeat proteins demonstrate its stable rigidity in the folded state, a massive 12× stretch ratio during unfolding, and a spontaneous refolding force that snaps the protein back to its original length with minimal energy dissipation.

Additionally, the Inventors also discovered that streptavidin (SAv) monomers incorporated into the presently claimed recombinant protein design self-associate into four-arm precursors, referred to as tetra-SAv, that are both stable and specific. Well-known for its strong protein-ligand binding affinity with biotin, streptavidin has been investigated for diverse biotechnology applications, including binding assays and purification strategies; however, it has never been used as a cross-linker for materials.

The goal of the present invention is to combine the defect-reducing and optimal crosslinking designs to develop a recombinant protein scaffold that mimics the massive reversible deformability of synthetic ankyrin repeats. The success of this project would be a novel and disruptive innovation, providing a significant improvement in durability, function, biocompatibility, and regenerative properties compared to the current synthetic cardiovascular tissues and implants on the market. Furthermore, this customizable biopolymer design will function as a platform technology that can incorporate recombinant proteins from other tissue types to develop an array of tissue-specific scaffolds that mimic natural mechanical properties and biological microenvironments. This biomimicry will improve the function and regenerative properties of products to deliver a massive improvement over current alternatives for hospitals, surgeons, and patients.

In some embodiments, the present invention features a method of modulating the mechanical properties (e.g., elasticity) of a biopolymer composition. The method may comprise solubilizing a plurality of polypeptide monomers (e.g., a polypeptide monomer composition) as described herein and adding a concentration of biotin or a derivative thereof to the solubilized polypeptide monomers. Solubilizing the polypeptide monomers allows for the first crosslinkers to associate together to form the biopolymer composition. In some embodiments, the concentration of biotin or derivative thereof modulates the mechanical properties (e.g., elasticity) of the biopolymer composition. In some embodiments, the concentration of biotin or a derivative thereof is about 0.01 mM to 10 mM.

The present invention may also feature a polypeptide monomer composition effective as a nano spring. The polypeptide monomer may comprise a first crosslinker (e.g., streptavidin polypeptide, an avidin polypeptide, or a derivative thereof) linked to a repeat component comprising a plurality of amino acid repeats linearly connected. In some embodiments, the polypeptide monomer comprises a first crosslinker (e.g., streptavidin polypeptide, an avidin polypeptide, or a derivative thereof), a secondary crosslinker linked together by a repeat component comprising a plurality of amino acid repeats linearly connected. In other embodiments, the polypeptide monomer comprises a first crosslinker (e.g., streptavidin polypeptide, an avidin polypeptide, or a derivative thereof) linked to a secondary crosslinker. In some embodiments, the polypeptide monomer comprises a repeat component linked to two secondary crosslinkers (e.g., one secondary crosslinker at an N-terminus and another secondary crosslinker at a C-terminus). In some embodiments, the polypeptide monomer further comprising biotin or a derivative thereof bound to the receptor of the first crosslinker.

In other embodiments, the present invention features a polypeptide monomer composition effective as a nano-spring. In some embodiments, the composition comprises a first crosslinker linked to an ankyrin component comprising a plurality of ankyrin repeats linearly connected. In other embodiments, the composition comprises a first crosslinker comprising a streptavidin protein linked to an ankyrin component comprising a plurality of ankyrin repeats linearly connected. In further embodiments, the composition comprises a first crosslinker comprising an avidin protein linked to an ankyrin component comprising a plurality of ankyrin repeats linearly connected.

In further embodiments, the present invention may also feature a method of producing a biopolymer composition. In some embodiments, the method comprises solubilizing a plurality of polypeptide monomers. In some embodiments, each monomer comprises a first crosslinker (e.g., a streptavidin polypeptide, an avidin polypeptide, or a derivative thereof), an ankyrin component comprising a plurality of ankyrin repeats linearly connected together linked to the first crosslinker, and a secondary crosslinker linked to the ankyrin component by a linker (e.g., a flexible linker). In some embodiments, solubilizing the polypeptide monomers allows for the first crosslinkers to associate together. In other embodiments, the method comprises activating the secondary crosslinkers such that the secondary crosslinkers associate together.

The present invention may further feature a biopolymer composition comprising a plurality of polypeptide monomers linked together. In some embodiments, each polypeptide monomer comprises a first crosslinker (e.g., a streptavidin polypeptide, an avidin polypeptide, or a derivative thereof), an ankyrin component comprising a plurality of ankyrin repeats linearly connected together and linked to the first crosslinker, and a secondary crosslinker linked to the ankyrin component by a linker (e.g., a flexible linker). In some embodiments, the streptavidin polypeptides associate together, and the secondary crosslinkers associate together.

One of the unique and inventive technical features of the present invention is using ankyrin repeat proteins as strands and streptavidin monomers (or avidin monomers) as cross-linkers. Without wishing to limit the invention to any theory or mechanism, it is believed that the technical feature of the present invention advantageously provides for a genetically engineered protein scaffold with a combination of biocompatibility, functionality, and bioactive regeneration. Furthermore, recombinant protein technology allows for the engineering of protein scaffold designs with precision at the amino acid level, promoting future customization of the present technology for specific tissue applications to increase the product line and capture a larger share of the market. None of the presently known prior references or work has the unique, inventive technical feature of the present invention.

Furthermore, the prior references teach away from the present invention. For example, current synthetic polymer scaffolds increase the patient's risk of a foreign body immune response, leading to a rejection of the implant, additional surgical interventions, and a lifetime of medications to prevent blood clotting.

Furthermore, the inventive technical features of the present invention contributed to a surprising result. For example, incorporating ankyrin repeat proteins as strands in polymer-network hydrogels improved the shear elastic modulus and relaxation time by three times compared to conventional flexible polymer strands. Additionally, streptavidin cross-linkers are associated with physical bonds; however, they exhibit extreme stability, displaying characteristics of permanent chemical bonds during rheology at low frequencies and elevated temperatures.

Another of the unique and inventive technical features of the present invention is controlling the elasticity of the hydrogel based on the amount of biotin in the solution. Structural changes in streptavidin can occur when biotin is bound to the protein receptors. However, there have not been any studies that demonstrate these structural changes impacting the mechanical behaviors of streptavidin when used as a cross-linker for hydrogels. Without wishing to limit the invention to any theory or mechanism, it is believed that the technical feature of the present invention advantageously allows for the modulation of a hydrogel's mechanical strength and stability when biotin binds to the receptor of streptavidin. None of the presently known prior references or work has the unique, inventive technical feature of the present invention.

Furthermore, the prior references teach away from the present invention. For example, currently, there are strategies to change the mechanical properties of hydrogels, such as adjusting the polymer concentration, length of the polymers, or types of cross-linkers.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1 shows a non-limiting example of the presently claimed genetically engineered recombinant protein that forms 4-arm streptavidin precursors (tetra-SAv). The recombinant protein comprises a first crosslinker (streptavidin monomer), ankyrin repeats, a flexible linker, and a secondary crosslinker. Once solubilized, the streptavidin monomers interact to form a tetramer with four arms.

FIG. 2 shows a non-limiting example of the presently claimed genetically engineered recombinant protein that forms 8-arm streptavidin precursors (octa-SAv). The recombinant protein comprises a first crosslinker (streptavidin monomer) between two strands containing ankyrin repeats, a flexible linker, and a secondary crosslinker. The streptavidin monomer is sandwiched on each end by an ankyrin repeat component, comprising a plurality of ankyrin repeats, a flexible linker, and a secondary crosslinker—this comprises one recombinant protein. Once solubilized, the streptavidin monomers interact to form a tetramer with eight arms.

FIGS. 3A and 3B show a non-limiting example of the presently claimed genetically engineered recombinant polypeptide biopolymer comprising a streptavidin tetramer with four arms, made using the recombinant protein from FIG. 1. FIG. 3A shows a recombinant polypeptide biopolymer comprising a first crosslinker (streptavidin tetramer), ankyrin repeats, a flexible linker, and a secondary crosslinker. FIG. 3B shows a recombinant polypeptide biopolymer comprising a first crosslinker (e.g., streptavidin tetramer), ankyrin repeats, a flexible linker, a secondary crosslinker, and four biotin molecules, each bound to one of the four streptavidin proteins comprising the tetramer.

FIGS. 4A and 4B show a non-limiting example of the presently claimed genetically engineered recombinant polypeptide biopolymer comprising a streptavidin tetramer with eight arms, made using the recombinant protein shown in FIG. 2. FIG. 4A shows a recombinant polypeptide biopolymer comprising a first crosslinker (streptavidin tetramer), ankyrin repeats, a flexible linker, and a secondary crosslinker. FIG. 4B shows a recombinant polypeptide biopolymer comprising a first crosslinker (streptavidin tetramer), ankyrin repeats, a flexible linker, a secondary crosslinker, and four biotin molecules. ** shows an example of a biotinylated protein/polymer/molecule/growth factor/drug/etc. that could be introduced to the presently claimed scaffold because of the strong interaction of biotin with streptavidin.

FIGS. 5A, 5B, 5C, 5D, and 5E show the biotin binding effect on the intermolecular strengths within SAv tetramer at a single-molecule level using nanoscale protein-based force probes fused SA tetramer. FIG. 5A shows a crystal structure of biotin (shown as van der Waals sphere)—SAv tetramer (PDB ID: 1SWE). FIG. 5B shows a schematic of the [(I27-SNase)₃-SAv monomer]₄ complex self-assembled through SAv monomers. FIG. 5C shows three AFM pulling geometries (F₁, F₂, and F₃) can be applicable when AFM stretches the SAv tetramer through the handles due to the connection of (I27-SNase)₃ handles to the N-terminus of each SAv monomer. Circles represent biotin binding sites (solid empty circle: front, dashed empty circle: back). FIG. 5D shows AFM force-extension curve of [(I27-SNase)₃-SAv monomer]₄. Multiple gray lines are worm-like chain (WLC) model that fits the curve with 44.5 nm of contour length increments (ΔLc) and 0.8 nm of persistent length (p), which corresponds to the unfolding of individual SNase domains. Dashed circles on the curve represent either rupture events of a single SAv tetramer or detachment events of protein handles from the AFM cantilever or the substrate. FIG. 5E shows probability densities of rupture forces, F_{final}, obtained from [(I27-SNase)₃-SAv monomer]₄ (solid curve; N=91) and of detachment forces, F_{detachment}, from I27-(SNase-I27)₃ handles alone (dashed curve; N=160) without biotin (top graph); and the probability densities for F_{final} of [(I27-SNase)₃-SAv monomer]₄ (solid curve; N=102) and F_{detachment} of I27-(SNase-I27)₃ (dashed curve; N=307) with 1 mM biotin (bottom graph). See FIGS. 6A and 6B for histogram results, FIG. 7B for the whole detachment force range, and FIG. 7C for the detachment results with 1 mM biotin in the buffer.

FIGS. 6A and 6B illustrate how to distinguish between the rupture forces of SAv tetramers and detachment forces in the presence of 0 mM (FIG. 6A) and 1 mM (FIG. 6B) biotin using the histogram method. [I27-(SNase-I27)3-SA]₄ and I27-(SNase-I27)₃ constructs were used to determine final forces with and without SAv tetramers at different biotin concentrations.

FIGS. 7A, 7B, and 7C show detachment events of I27-(SNase-I27)₃ force probes. FIG. 7A shows an AFM force-extension curve of I27-(SNase-I27)₃ shows mechanical unfolding fingerprints of SNase modules with 42 nm of contour length increments (ΔLc) and 0.8 nm of persistent length (p) and 127 domains with 30 nm of ΔLc and 0.35 nm of p. ΔLc and p are obtained by applying multiple worm-like chain (WLC) fits on the curve (multiple gray lines). The final force peak on the curve (gray dotted circle) represents the detachment force of an I27-(SNase-I27)₃ molecule either from the substrate or the AFM cantilever (gray dotted circles in the inset of FIGS. 7A and 7B). FIGS. 7A and 7B show probability densities of I27-(SNase-I27)₃ detachment forces with 0 mM biotin in B (N=160) and 1 mM biotin in C (N=307) by kernel density estimation (KDE; curved line) and histogram (grey line).

FIGS. 8A, 8B, and 8C shows schematics and SDS-PAGE analysis of purified artificial protein constructs used for fabricating SAv-based hydrogels. FIG. 8A shows SAv tetramer with SpyTag (ST) genetically fused to the C-terminus of each SAv monomer (SAv monomer-ST)₄. The SDS-PAGE analysis illustrates the purity of lyophilized protein samples and the self-assembly capability of SAv. Lane 1 shows SAv tetramers denatured to monomers by boiling the samples at 100° C. Lane 2 depicts the native conditions leading to SAv tetramer formation, demonstrating the successful self-assembly of SAv post biosynthesis. FIG. 8B shows SpyCatcher (SC) was genetically fused at each terminus of a flexible, intrinsically disordered protein, known as C₂₄ (SC-C₂₄-SC). The SDS-PAGE analysis shows high sample purity and slightly higher relative molecular weight (M.W.) than the theoretical M.W. However, LC/MS analysis reveals that the sample M.W. matches the theoretical M.W. (Table 1). The higher relative M.W. observed on SDS-PAGE is attributed to the presence of C₂₄ protein. FIG. 8C shows that by combining these constructs at a final 1:1 molar ratio between ST and SC, a polymer network was self-assembled and characterized with different concentrations of biotin (represented by gold dots).

FIGS. 9A, 9B, and 9C shows visual inspection and rheological characterization of FIG. 9A SAv-based hydrogels with and without biotin. FIG. 9B shows representative strain sweeps for SAv-based hydrogel samples with different biotin concentrations. FIG. 9C shows experimental G′ with different biotin concentration (N=5; **p<0.01; ****p<0.0001; ns=no significance; see Table 2).

FIGS. 10A and 10B shows representative frequency sweep of SAv-based hydrogel samples with SC-C₂₄-SC (FIG. 10A) or SC-NI₆C-SC (FIG. 10A) constructs from 0 to 10 mM biotin. The lack of crossover frequencies (G′=G″) demonstrate that physically crosslinked SAv tetramers act as a chemical cross-linker.

FIG. 11 shows strain sweeps of hydrogels with SC-C₂₄-SC or SC-NI₆C-SC constructs at different biotin concentrations.

FIG. 12A shows physically cross-linked hydrogel with P coiled-coil cross-linkers.

FIG. 12B shows a box-and-whisker plot of a P-cross-linked hydrogel at different biotin concentrations (represented by gold dots in schematic FIG. 13A) showing no significant changes in experimental shear elastic modulus (G′) without any SAv tetramer cross-linkers present (one way ANOVA; F(3, 18)=2.232; p=0.1195; ns=no significance; 0 mM and 1 mM, N=6; 0.5 mM and 10 mM, N=5).

FIGS. 13A and 13B shows a schematic of an SA-based polymer network with SC-NI₆C-SC constructs (FIG. 13A). FIG. 13B shows an SDS-PAGE analysis of SC-NI₆C-SC displays the purity of protein samples used to fabricate SAv-based hydrogels.

FIG. 14A shows representative strain sweeps of SAv-based hydrogel samples with SC-NI₆C-SC.

FIG. 14B shows a bar graph of experimental shear G′ (see Table 1; **p<0.01; ***p<0.0005)

FIGS. 15A and 15B shows a bioerosion experiment of SAv-based hydrogel in the presence and absence of SAv ligand, biotin. FIG. 15A shows a schematic of bioerosion tests and a polymer network of hydrogels. All bioerosion experiments were performed with a 1:9 volume ratio of the hydrogel:solvent, with or without biotin (gold dots) at 37° C. FIG. 15B shows a bioerosion graph depicting erosion rates over time (slope k; eroded hydrogel %/hour). Simple linear regression analyses were conducted for the following time intervals: 0-4 hours (dotted line) and 4-48 hours (solid line). The sample size of each time point was in triplicate (N=3).

FIG. 16 shows SDS-PAGE analysis of (un)denatured SAv-ST₄ proteins used in hydrogel fabrication at various biotin concentrations. The result demonstrates that all the SAv tetramers remain intact even after boiling at 100° C. (denoted as +) in the presence of 10 mM biotin. This observation indirectly suggests that a biotin concentration of 10 mM is sufficient enough to provide mechanical reinforcement to every SAv tetramer within the hydrogel.

FIGS. 17A and 17B shows additional bioerosion tests with simple linear regression curves of SAv-based hydrogels (N=3). FIG. 17A shows bioerosion rates at room temperature significantly decreased where the half-life of the hydrogel sample was 329.76 hours, a ˜230% increase compared to the half-life at 37° C. (99.55 hours). FIG. 17B shows a comparison of bioerosion rates between SAv-based hydrogels and P coiled-coil cross-linked hydrogels (Figure S6) at 25° C. revealed a half-life of approximately 329.76 hours for SAv-containing hydrogels and 2.6 hours for hydrogels without SAv tetramers.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

The present invention is entirely composed of biocompatible recombinant proteins that biodegrade into amino acid byproducts in a natural process that recycles or disposes of the scaffold without risk of harmful side effects. This technology also contains protein sequences, such as the Arginine-Glycine-Aspartate (RGD) motifs that promote cell adhesion and other growth factors that promote cell adhesion, migration, and proliferation. Furthermore, the mechanical properties of the present invention will mimic natural tissue properties to maintain function after implantation and promote cell viability for specific cell types associated with the tissue type. These factors will allow the presently claimed composition to be functional post-implantation, then initiate cellular remodeling of the scaffold to naturally degrade the recombinant protein scaffold and regenerate natural tissue in its place. The improvement in the performance of these scaffolds will significantly disrupt the current market and provide a new gold standard of care for treating cardiovascular disease.

The terms “peptide,” “polypeptide,” and “protein” are used interchangeably herein and refer to any molecule that includes at least 2 or more amino acids.

Referring now to the figures, the present invention features a biopolymer composition that is effective as a nanospring. In some embodiments, the composition comprises a first crosslinker linked to an ankyrin component comprising a plurality of ankyrin repeats linearly connected to each other. In some embodiments, compositions described herein may be used to produce springy protein-based materials.

The present invention may also feature a method of modulating mechanical properties (e.g., elasticity) of a biopolymer composition. The method may comprise solubilizing a plurality of polypeptide monomers as described herein and adding a concentration of biotin or a derivative thereof to the solubilized polypeptide monomers. Solubilizing the polypeptide monomers allows for the first crosslinkers to associate together to form the biopolymer composition. In some embodiments, the concentration of biotin or a derivative thereof modulates the mechanical properties of the biopolymer composition.

In some embodiments, solubilizing the polypeptide monomers allows for the first crosslinker to self-assemble into a tetramer. Optionally, the secondary crosslinker may then form bonds with each other.

In some embodiments, the method may comprise solubilizing a plurality of two or more polypeptide monomers (e.g., pairs of polypeptide monomers) as described herein and adding a concentration of biotin or a derivative thereof to the solubilized polypeptide monomers. In other embodiments, the method may comprise solubilizing a plurality of two polypeptide monomers as described herein and adding a concentration of biotin or a derivative thereof to the solubilized polypeptide monomers. Solubilizing two polypeptide monomers allows for the first crosslinkers and/or secondary crosslinkers to associate together to form the biopolymer composition.

In some embodiments, the concentration of biotin or a derivative thereof is about 0.01 mM to 10 mM. In some embodiments, the concentration of biotin or a derivative thereof is about 0.01 mM to 10 mM, or about 0.01 mM to 5 mM, or about 0.01 to 2 mM, or about 0.01 mM to 1 mM, or about 0.01 mM to 0.5 mM, or about 0.01 mM to 0.25 mM, or about 0.01 mM to 0.1 mM. In some embodiments, the concentration of biotin or a derivative thereof is about 0.1 mM to 10 mM, or about 0.1 mM to 5 mM, or about 0.1 to 2 mM, or about 0.1 mM to 1 mM, or about 0.1 mM to 0.5 mM, or about 0.1 mM to 0.25 mM. In some embodiments, the concentration of biotin or a derivative thereof is about 0.25 mM to 10 mM, or about 0.25 mM to 5 mM, or about 0.25 to 2 mM, or about 0.25 mM to 1 mM, or about 0.25 mM to 0.5 mM. In some embodiments, the concentration of biotin or a derivative thereof is about 0.5 mM to 10 mM, or about 0.5 mM to 5 mM, or about 0.5 to 2 mM, or about 0.5 mM to 1 mM, or about 1 mM to 10 mM, or about 1 mM to 5 mM, or about 1 to 2 mM, or about 2 mM to 10 mM, or about 2 mM to 5 mM, or about 5 mM to 10 mM.

The present invention may also feature a polypeptide monomer composition effective as a nano spring. The polypeptide monomer may comprise a first crosslinker (e.g., streptavidin polypeptide, an avidin polypeptide, or a derivative thereof) linked to a repeat component comprising a plurality of amino acid repeats linearly connected.

In some embodiments, the polypeptide monomer comprises a first crosslinker (e.g., streptavidin polypeptide, an avidin polypeptide, or a derivative thereof), a secondary crosslinker linked together by a repeat component comprising a plurality of amino acid repeats linearly connected. For example, the polypeptide monomer comprises (N)First Crosslinker(C), (N)Repeat Component(C), (N)Secondary crosslinker(C); where (N) represents the N-terminal and (C) represents the C-terminal of the respective protein sequences. Non-limiting examples of polypeptide monomers include but are not limited to:
(N)First Crosslinker(C)-(N)Repeat Component(C)-(N)Secondary Crosslinker(C)
(N)First Crosslinker(C)-(N)Ankyrin Component(C)-(N)Secondary Crosslinker(C)
(N)First Crosslinker(C)-(N)Ankyrin Component(C)-(N)GB1A(C)
(N)First Crosslinker(C)-(N)Ankyrin Component(C)-(N)GB1B(C)
(N)First Crosslinker(C)-(N)Ankyrin Component(C)-(N)SpyCatcher(C)
(N)First Crosslinker(C)-(N)Ankyrin Component(C)-(N)SpyTag(C)
(N)First Crosslinker(C)-(N)C₂₄ (SEQ ID NO: 6)(C)-(N)Secondary Crosslinker(C)
(N)First Crosslinker(C)-(N)C₂₄ (SEQ ID NO: 6)(C)-(N)GB1A(C)
(N)First Crosslinker(C)-(N)C₂₄ (SEQ ID NO: 6)(C)-(N)GB1B(C)
(N)First Crosslinker(C)-(N)C₂₄ (SEQ ID NO: 6)(C)-(N)SpyCatcher(C)
(N)First Crosslinker(C)-(N)C₂₄ (SEQ ID NO: 6)(C)-(N)SpyTag(C)
(N)First Crosslinker(C)-(N)NI₆C (SEQ ID NO: 7)(C)-(N)Secondary Crosslinker(C)
(N)First Crosslinker(C)-(N)NI₆C (SEQ ID NO: 7)(C)-(N)GB1A(C)
(N)First Crosslinker(C)-(N)NI₆C (SEQ ID NO: 7)(C)-(N)GB1B(C)
(N)First Crosslinker(C)-(N)NI₆C (SEQ ID NO: 7)(C)-(N)SpyCatcher(C)
(N)First Crosslinker(C)-(N)NI₆C (SEQ ID NO: 7)(C)-(N)SpyTag(C)

In other embodiments, the polypeptide monomer comprises a first crosslinker (e.g., streptavidin polypeptide, an avidin polypeptide, or a derivative thereof) linked to a secondary crosslinker. For example, the polypeptide monomer may comprise a first crosslinker e.g., streptavidin polypeptide, an avidin polypeptide, or a derivative thereof) linked to either a GB1A or a GB1B. Alternatively, the polypeptide monomer may comprise a first crosslinker e.g., streptavidin polypeptide, an avidin polypeptide, or a derivative thereof) linked to either a SpyCatcher or a SpyTag. Non-limiting examples of polypeptide monomers include but are not limited to:
(N)First Crosslinker(C)-(N)Secondary Crosslinker(C)
(N)First Crosslinker(C)-(N)GB1A(C)
(N)First Crosslinker(C)-(N)GB1B(C)
(N)First Crosslinker(C)-(N)SpyCatcher(C)
(N)First Crosslinker(C)-(N)SpyTag(C)

In other embodiments, the polypeptide monomers may comprise a first crosslinker tetramer (e.g., a streptavidin tetramer, an avidin tetramer, or a derivative thereof) where each monomer of the first crosslinker tetramer is linked to a secondary crosslinker (e.g., SpyTag or GB1A).

In some embodiments, the polypeptide monomer comprises a repeat component linked to two secondary crosslinkers (e.g., one secondary crosslinker at an N-terminus and another secondary crosslinker at a C-terminus; e.g., SEQ ID NO: 2 and SEQ ID NO: 3). Non-limiting examples of polypeptide monomers include but are not limited to:
(N)Secondary Crosslinker(C)-(N)Repeat Component(C)-(N)Secondary Crosslinker(C)
(N)GB1A(C)-(N)Repeat Component(C)-(N)GB1A(C)
(N)GB1B(C)-(N)Repeat Component(C)-(N)GB1B(C)
(N)SpyCatcher(C)-(N)Repeat Component(C)-(N)SpyCatcher(C)
(N)SpyTag(C)-(N)Repeat Component(C)-(N)SpyTag(C)

In some embodiments, solubilizing polypeptide monomer comprising a secondary crosslinker allows for one secondary crosslinker (e.g., GB1A or SpyCatcher) to associate with a corresponding secondary crosslinker (e.g., GB1B or SpyTag) to form the biopolymer composition.

Non-limiting examples of polypeptide monomer pairs include but are not limited to:

(N)First Crosslinker(C)-(N)Secondary Crosslinker(C) +
(N)Secondary Crosslinker(C)-(N)Repeat Component(C)-
(N)Secondary Crosslinker(C)
(N)First Crosslinker(C)-(N)GB1A(C) +
(N)GB1B(C)-(N)Repeat Component(C)-(N)GB1B(C)
(N)First Crosslinker(C)-(N)GB1B(C) +
(N)GB1A(C)-(N)Repeat Component(C)-(N)GB1A(C)
(N)First Crosslinker(C)-(N)SpyCatcher(C) +
(N)SpyTag(C)-(N)Repeat Component(C)-(N)SpyTag(C)
(N)First Crosslinker(C)-(N)SpyTag(C) +
(N)SpyCatcher(C)-(N)Repeat Component(C)-(N)SpyCatcher(C)

The aforementioned polypeptide monomer may further comprise biotin or a derivative thereof bound to the receptor of the first crosslinker.

Methods described herein may also comprise solubilizing i) a plurality of polypeptide monomers comprising a first crosslinker tetramer and ii) a plurality of polypeptide monomers comprising a repeat component linked to two secondary crosslinkers (e.g., SEQ ID NO: 2 or SEQ ID NO: 3) and adding a concentration of biotin or a derivative thereof to the solubilized polypeptide monomers. Solubilizing two polypeptide monomers allows for the secondary crosslinkers to associate together to form the biopolymer composition.

In some embodiments, the repeat component comprises an ankyrin component comprising a plurality of ankyrin repeats linearly connected together. In some embodiments, the repeat component comprises an ankyrin component comprising a plurality of ankyrin repeats linearly connected together linked to the first crosslinker. In other embodiments, the repeat component comprises SEQ ID NO: 6 or SEQ ID NO: 7. In some embodiments, the repeat component comprises SEQ ID NO: 6 linked to the first crosslinker. In some embodiments, the repeat component comprises SEQ ID NO: 7 linked to the first crosslinker.

In some embodiments, the first crosslinker comprises a biotin-binding protein, including but not limited to streptavidin, avidin, or a derivative thereof. In some embodiments, the first crosslinker comprises a streptavidin polypeptide or a derivative thereof. In other embodiments, the first crosslinker comprises an avidin polypeptide or a derivative thereof.

In some embodiments, the present invention may also feature a polypeptide monomer (i.e., an individual polypeptide) composition comprising a first crosslinker. In some embodiments, the composition comprises an ankyrin component comprising a plurality of ankyrin repeats linearly connected together linked to the first crosslinker. In other embodiments, the composition comprises a secondary crosslinker linked to the ankyrin component by a flexible linker.

In some embodiments, the ankyrin component is linked to the C-terminal of the first crosslinker. In some embodiments, the flexible linker is connected to the C-terminal of the ankyrin component. In some embodiments, the secondary crosslinker is linked to the C-terminal of the flexible linker. In other embodiments, the biopolymer composition described herein may comprise a protein in which the first crosslinker is at the N-terminal of the biopolymer composition and the secondary crosslinker is at the C-terminal of the biopolymer composition. For example, a protein comprises (N)First Crosslinker(C), (N)Ankyrin(C), (N)Flexible Linker(C), (N)Secondary crosslinker(C); where (N) represents the N-terminal and (C) represents the C-terminal of the respective protein sequences.

In other embodiments, the flexible linker is linked to the C-terminal of the secondary crosslinker. In some embodiments, the ankyrin component is linked to the C-terminal of the flexible linker. In some embodiments, the first crosslinker is linked to the C-terminal of the ankyrin component. In some embodiments, the biopolymer composition described herein may comprise a protein in which the secondary crosslinker is at the N-terminal of the biopolymer composition and the first crosslinker is at the C-terminal of the biopolymer composition. For example, the protein may comprise (N)Secondary Crosslinker(C), (N)Flexible Linker(C), (N)Ankyrin(C), (N)First crosslinker(C); where (N) represents the N-terminal and (C) represents the C-terminal of the respective protein sequences.

In some embodiments, the secondary crosslinker is a chemical crosslinker, including but not limited to GB1. In some embodiments, the secondary crosslinker comprises either a GB1A or GB1B. In other embodiments, the secondary crosslinker comprises either a SpyCatcher or SpyTag. As used herein, a “chemical crosslinker” may refer to a crosslinker in which two or more subcomponents recognize and bind to each other with covalent bonds (i.e., permanent bonds). In some embodiments, the chemical crosslinker is a photocrosslinker. In other embodiments, the secondary crosslinker is a photocrosslinker, such as Tyrosine.

In other embodiments, the secondary crosslinker is a physical crosslinker, including but not limited to Cartilage Oligomeric Matrix Protein. As used herein, a “physical crosslinker” may refer to a crosslinker in which two or more subcomponents recognize and bind each other non-covalently (e.g., with hydrogen bonds, hydrophobic interactions, and others that are not chemical bonds). In some embodiments, the secondary crosslinker promotes the ankyrin repeats mechanics.

As used herein, “ankyrin repeat mechanics” refers to the unfolding of the tertiary protein structure and forceful refolding back to its original conformation with minimal energy dissipation, acting as a nano-spring.

As used herein, a “nano-spring” refers to a tertiary protein structure that, when an external force is applied, unfolds at the threshold force and can extend in length (e.g., up to 12× times its original length). When the external force is below the threshold force, the tertiary protein structure refolds back to its original structure (e.g., its original length). In some embodiments, the folding time (i.e., the unfolding/refolding time) is less than 1 second. In other embodiments, the folding time (i.e., the unfolding/refolding time) is less than 1 millisecond.

In some embodiments, the ankyrin component described herein is a nanospring. In some embodiments, the ankyrin component can expand up to 12× its original length when a force is applied to said component. In some embodiments, the ankyrin component described herein is a linear nanospring. In other embodiments, the ankyrin component described herein is a non-linear nanospring.

Without wishing to limit the present invention to any theory or mechanism, it is thought that secondary crosslinkers may promote ankyrin repeat protein mechanics after forming the polymer-network hydrogel scaffold (e.g., the biopolymer composition disclosed herein). The promotion of the ankyrin repeat protein mechanics may be because of 1) the strength of the secondary cross-linker and 2) the ability of the secondary cross-linker to form a homogeneous biopolymer network.

Without wishing to limit the present invention to any theory or mechanism, it is believed that the secondary crosslinker (e.g., GB1) self-associates through physical interactions before forming chemical cysteine bonds. Dithiothreitol (DTT) is added to the solution to prevent GB1 from forming cysteine bonds. When DTT is removed or degraded in the solution, cysteine bonds can form. Without wishing to limit the present invention to any theory or mechanism, it is believed that this process allows for the reorganization of the biopolymer network before permanent bonds are formed to promote the formation of a homogeneous distribution of biopolymers within the network.

In some embodiments, the composition comprises a biotin molecule bound to the first crosslinker. In other embodiments, the composition comprises two biotin molecules bound to the first crosslinker. In some embodiments, the composition comprises three biotin molecules bound to the first crosslinker. In other embodiments, the composition comprises four biotin molecules bound to the first crosslinker. In some embodiments, the composition comprises biotin or a derivative thereof bound to the first crosslinker. In other embodiments, the composition comprises two biotins or derivatives thereof bound to the first crosslinker. In some embodiments, the composition comprises three biotins or derivatives thereof bound to the first crosslinker. In other embodiments, the composition comprises four biotins or derivatives thereof bound to the first crosslinker.

Without wishing to limit the present invention to any theory or mechanism, it is believed that biotin has a high affinity to streptavidin, which allows it to bind to the first crosslinker. In some embodiments, the biotin molecule comprises a protein, polymer, or molecule covalently attached to biotin using a biotinylation process. In other embodiments, the composition comprises a secondary cross-linker connected to the protein, polymer, or molecule covalently attached to biotin using a biotinylation process.

As used herein, “a biotinylation process” refers to the process of covalently attaching biotin to a protein, nucleic acid, and/or other molecules. Biotinylation is rapid and specific and is unlikely to disturb the natural function of the molecule due to the small size of biotin.

In other embodiments, the biotin molecule further comprises a biotinylated protein, polymer, biopolymer, molecule, growth factor, drug, etc., linked by a linker. In some embodiments, the biotin molecule further comprises a secondary crosslinker linked by a linker. In other embodiments, the composition further comprises a biotinylated protein bound to the first crosslinker.

In some embodiments, the compositions described herein comprise a linker. In some embodiments, a linker may be used to connect a secondary crosslinker to the composition described herein. For example, a linker may be used to connect a secondary crosslinker to an ankyrin component and/or a linker may be used to connect a secondary crosslinker to a biotin molecule.

In some embodiments, the linker is protein based. In other embodiments, the linker is a flexible linker. In some embodiments, the flexible linker is protein based. In some embodiments, the linker is flexible. In other embodiments, the linker is rigid. In further embodiments, the linker is a combination of flexible and rigid. In some embodiments, the combination of flexible and rigid controls the flexibility of the strand. In some embodiments, the combination of flexible and rigid controls the flexibility of the strand between the biotin and the secondary cross-linker. In some embodiments, the linker has no structure. In preferred embodiments, the linker has hydrophilic properties. In further embodiments, the linker comprises structured protein (e.g., ankyrin).

In some embodiments, the polypeptide monomer composition is used for drug delivery. In other embodiments, the polypeptide monomer composition is used as a nano or micron-sized drug delivery system. In further embodiments, the polypeptide monomer composition is used for imaging applications. In some embodiments, the drug is bound to the polypeptide monomer composition via biotin. In other embodiments, a growth factor may be bound to the polypeptide monomer composition via biotin. In further embodiments, the polypeptide monomer composition may be used for tissue engineering, including but not limited to vascular tissue (e.g., a cardiac patch for the heart), blood vessels (e.g., arteries or veins), soft tissues (e.g., muscles or cartilage), or elastic tissues (e.g., skin tissues). In other embodiments, the polypeptide monomer composition may be utilized in high temperature applications.

In some embodiments, the polypeptide monomer composition comprises an ankyrin component comprising about 2 to 24 ankyrin repeats. In other embodiments, the polypeptide monomer composition comprises an ankyrin component comprising about 3 to 24 ankyrin repeats. In some embodiments, the ankyrin component comprises 2 to 24 ankyrin repeats, or 2 to 18 ankyrin repeats, or 2 to 12 ankyrin repeats, or 2 to 6 ankyrin repeats, or 3 to 24 ankyrin repeats, or 3 to 18 ankyrin repeats, or 3 to 12 ankyrin repeats, or 3 to 6 ankyrin repeats, or 6 to 24 ankyrin repeats, or 6 to 18 ankyrin repeats, or 6 to 12 ankyrin repeats, or 12 to 24 ankyrin repeats, or 12 to 18 ankyrin repeats or 18 to 24 ankyrin repeats. In some embodiments, the ankyrin component comprises 2 ankyrin repeats, 3 ankyrin repeats, 4 ankyrin repeats, or 5 ankyrin repeats. In other embodiments, the ankyrin component comprises 6 ankyrin repeats, 7 ankyrin repeats, 8 ankyrin repeats, 9 ankyrin repeats, or 10 ankyrin repeats. In some embodiments, the ankyrin component comprises 11 ankyrin repeats, 12 ankyrin repeats, 13 ankyrin repeats, 14 ankyrin repeats, or 15 ankyrin repeats. In other embodiments, the ankyrin component comprises 16 ankyrin repeats, 17 ankyrin repeats, 18 ankyrin repeats, 19 ankyrin repeats, or 20 ankyrin repeats. In some embodiments, the ankyrin component comprises 21 ankyrin repeats, 22 ankyrin repeats, 23 ankyrin repeats, or 24 ankyrin repeats.

As used herein, an “ankyrin repeat” may refer to a recombinant protein or polypeptide comprising of two or more ankyrin protein sequences, including either the complete ankyrin protein sequence or a partial ankyrin protein sequence comprising one or more domains of the entire ankyrin protein sequence. In some embodiments, a partial ankyrin protein comprises at least two ankyrin repeats (e.g., repeat domains). Without wishing to limit the present invention to any theory or mechanism, it is believed that at least two ankyrin repeats (e.g., repeat domains) are required to prepare an ankyrin component comprising mechanical or nanospring properties.

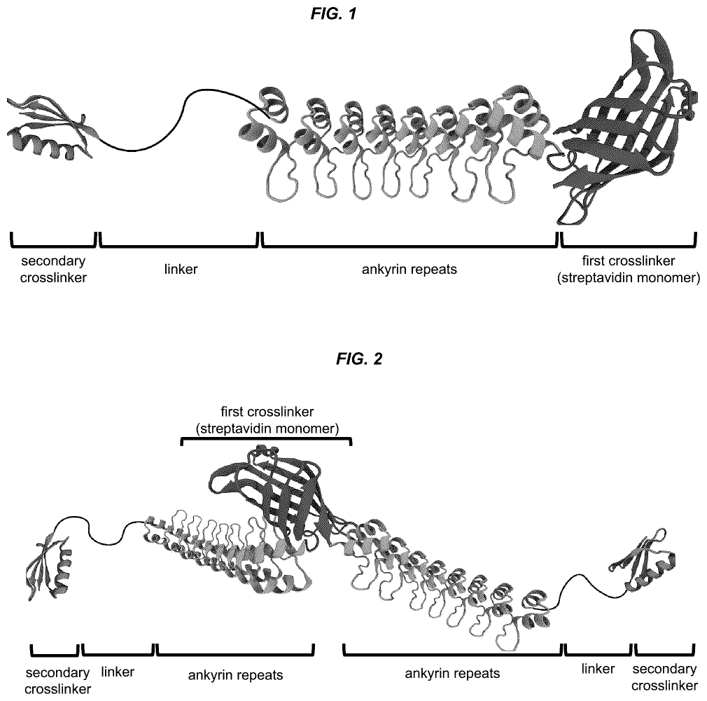

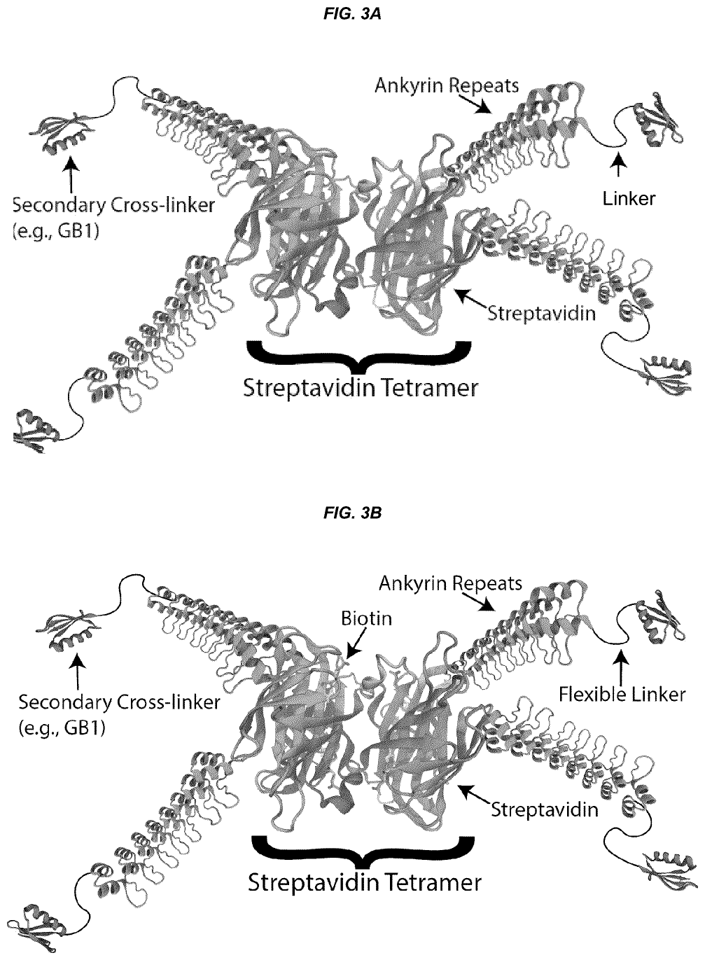

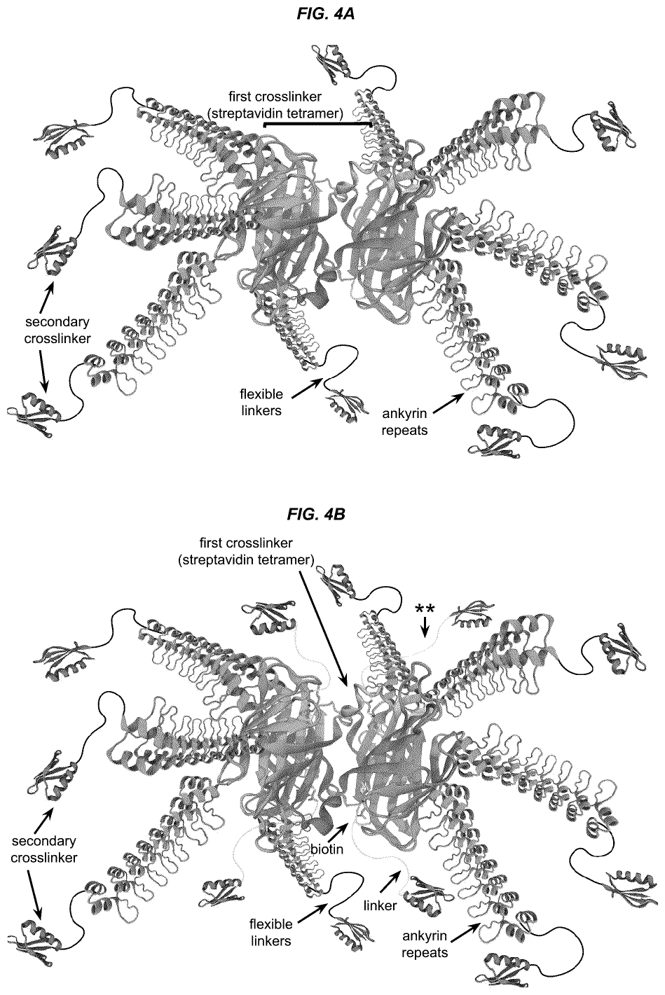

In preferred embodiments, the biopolymer composition comprises 4 arms projecting from each streptavidin tetramer (i.e., tetra-SAv, (see FIG. 3A)). In some embodiments, the biopolymer composition comprises 1 arm projecting from one streptavidin monomer (FIG. 1). In some embodiments, the biopolymer composition comprises two arms projecting from one streptavidin monomer (FIG. 2). In some embodiments, the biopolymer composition comprises 8 arms projecting from each streptavidin tetramer (FIG. 4A). In some embodiments, the biopolymer composition comprises up to 12 arms projecting from each streptavidin tetramer. In some embodiments, the biopolymer composition comprises 9 arms, or 10 arms, or 11 arms, or 12 arms projecting from each streptavidin tetramer

In some embodiments, biotin is used to create a biopolymer composition comprising 12 arms projecting from each streptavidin tetramer.

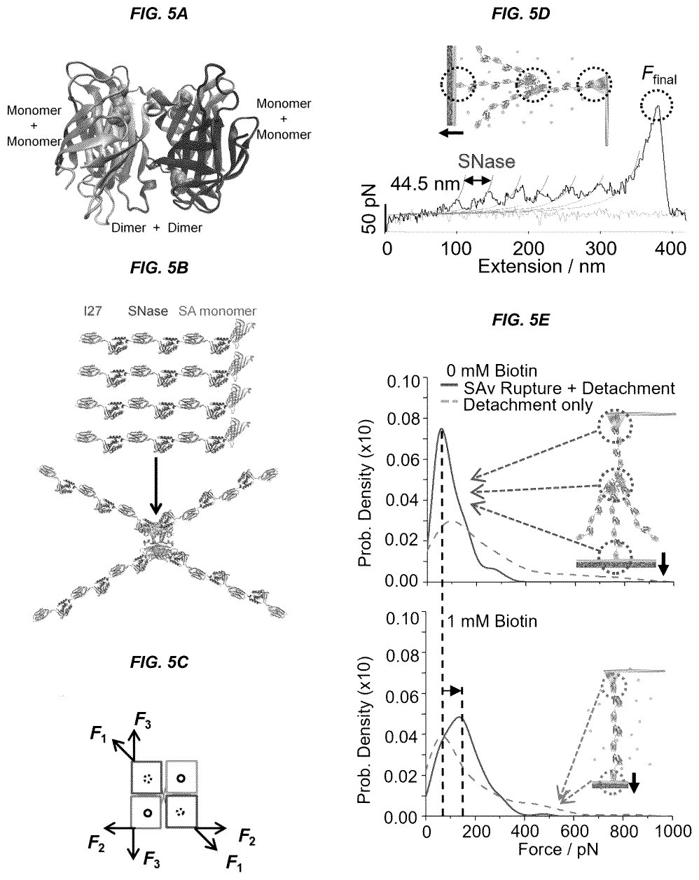

Without wishing to limit the present invention to any theory or mechanism, it is believed that the SAv tetramer is self-assembled from four identical SAv monomers. The monomers bind tightly to each other, forming dimers, which then self-associate with somewhat less tightness compared to the monomer interactions, ultimately forming tetramers (FIG. 5A). This results in the SAv tetramer containing two monomer-monomer interfaces and one dimer-dimer interface.

As used herein, a “streptavidin precursor” may refer to a four- (FIG. 3A), eight- (FIG. 4A), or twelve- (FIG. 4B) arm macromolecule assembly that forms when streptavidin within recombinant proteins from FIG. 1 or FIG. 2 interact to form tetramers and, in some cases, biotinylated secondary cross-linkers are introduced to the macromolecule assembly. In some embodiments, the streptavidin precursor binds together to form the hydrogel scaffold.

As used herein, the term “arm” refers to the number of linkers extended from a first crosslinker described herein (e.g., a streptavidin precursor (see FIG. 1 or 2) or a streptavidin tetramer (see FIGS. 3A and 3B or FIGS. 4A and 4B), or an avidin precursor or an avidin tetramer) In some embodiments, the linkers may comprise a secondary crosslinker, proteins, polymers, or molecule (e.g., macromolecules).

The present invention may also feature a method of producing a polypeptide biopolymer composition. In some embodiments, the method comprises solubilizing a plurality of polypeptide monomers, as described herein. In some embodiments, each polypeptide monomer (i.e., an individual polypeptide) comprises a first crosslinker (e.g., a streptavidin polypeptide), an ankyrin component comprising a plurality of ankyrin repeats linearly connected together linked to the first crosslinker, and a secondary crosslinker linked to the ankyrin component by a linker (e.g., a flexible linker). In some embodiments, solubilizing the polypeptide monomers (i.e., an individual polypeptide) allows the first crosslinkers (e.g., a streptavidin polypeptide) to associate together. In other embodiments, the method comprises activating the secondary crosslinkers such that the secondary crosslinkers associate together.

In further embodiments, the method may further comprise adding biotin before activating the secondary crosslinkers. In other embodiments, the method may further comprise adding biotin after activating the secondary crosslinkers. In some embodiments, the biotin binds to the first crosslinker. In some embodiments, the method further comprises adding a biotinylated protein before activating the secondary crosslinkers. In some embodiments, the method further comprises adding a biotinylated protein after activating the secondary crosslinkers. In some embodiments, the biotinylated protein binds to the first crosslinker. In further embodiments, a biotinylated protein, polymer, molecule, growth factor, drug, etc., is added before activating the secondary crosslinkers.

The present invention may feature a biopolymer composition comprising a plurality of polypeptide monomers linked together. In some embodiments, each polypeptide monomer comprises a first crosslinker (e.g., a streptavidin polypeptide), an ankyrin component comprising a plurality of ankyrin repeats linearly connected together linked to the first crosslinker, and a secondary crosslinker linked to the ankyrin component by a linker (e.g., a flexible linker). In some embodiments, the first crosslinkers (e.g., a streptavidin polypeptide) associate together, and the secondary crosslinkers associate together.

In some embodiments, the biopolymer composition is solubilized in water. In some embodiments, the biopolymer composition is solubilized in a phosphate-buffered solution. In some embodiments, the biopolymer composition is solubilized in a tris-buffered solution. In some embodiments, the biopolymer composition is solubilized in a saline solution, including but not limited to 0.9% sodium chloride. In some embodiments, the biopolymer composition is solubilized in a Lactated Ringers solution, including but not limited to sodium chloride, potassium chloride, calcium chloride, and sodium lactate. In some embodiments, the biopolymer composition is solubilized in a sugar solution, including but not limited to 5% dextrose.

In some embodiments, the biopolymer composition is solubilized at room temperature. In some embodiments, the biopolymer composition is solubilized between about 2° C. to 40° C. In some embodiments, the biopolymer composition is solubilized at about 4° C. In some embodiments, the biopolymer composition is solubilized at about 10° C. In some embodiments, the biopolymer composition is solubilized at about 20° C. In some embodiments, the biopolymer composition is solubilized at about 30° C. In some embodiments, the biopolymer composition is solubilized at about 35° C. In some embodiments, the biopolymer composition is solubilized at about 37° C. In some embodiments, the biopolymer composition is solubilized at about 40° C.

In some embodiments, the secondary cross-linker is a chemical crosslinker, including but not limited to GB1. In some embodiments, activating GB1 comprises removing DTT. In other embodiments, activating a chemical crosslinker comprises introducing energy into the system to form the bond. Non-limiting examples of introducing energy into the system may include but are not limited to the use of specific wavelengths of light (via photocrosslinking) or a particular temperature (i.e., thermal crosslinking). In other embodiments, activating a chemical crosslinker may comprise adding chemicals to the solution, such as but not limited to a free radical. In other embodiments, some chemical cross-linkers are spontaneous and do not require activation. For example, a SpyTag-SpyCatcher secondary crosslinking system, which are two separate proteins that recognize and interact to form a spontaneous peptide bond.

In other embodiments, the secondary crosslinker is a physical crosslinker. In some embodiments, physical crosslinkers do not need to be activated. In other embodiments, physical crosslinkers are attracted by non-covalent bonds, including but not limited to hydrophobic bonds, hydrogen bonds, van der Waals interactions, or ionic bonds. In some embodiments, these aforementioned bonds may be inactivated by changing the pH of the buffer or by adding specific solutes or chemicals.

In some embodiments, the secondary crosslinker is a photocrosslinker. In some embodiments, activating a photocrosslinker comprises exposing the solution to a specific wavelength of light. In some embodiments, activating a photocrosslinker comprises exposing the solution to a broad-spectrum light source. In some embodiments, the photocrosslinker comprises a tyrosine residue crosslinker. In some embodiments, the tyrosine residue crosslinker is activated by adding a ruthenium and an ammonium persulfate catalyst and exposing the crosslinker to a light, therefore forming a dityrosine. In some embodiments, the crosslinker is exposed to a blue light. In other embodiments, the crosslinker is exposed to UV light. In other embodiments, the tyrosine residue crosslinker is activated by adding a ruthenium and an ammonium persulfate catalyst and exposing the crosslinker to blue light. In further embodiments, the tyrosine residue crosslinker is activated by adding ruthenium and an ammonium persulfate catalyst and exposing the crosslinker to UV light.

In some embodiments, the tyrosine residue crosslinker is activated by adding a riboflavin (i.e., vitamin B2) and an ammonium persulfate catalyst and exposing the crosslinker to a light, therefore forming a dityrosine. In some embodiments, the crosslinker is exposed to a blue light. In other embodiments, the crosslinker is exposed to UV light. In other embodiments, the tyrosine residue crosslinker is activated by adding riboflavin (i.e., vitamin B2) and an ammonium persulfate catalyst and exposing the crosslinker to a blue light. In further embodiments, the tyrosine residue crosslinker is activated by adding riboflavin (i.e., vitamin B2) and an ammonium persulfate catalyst and exposing the crosslinker to UV light.

In some embodiments, adding ruthenium and an ammonium persulfate catalyst is required to activate the tyrosine residue crosslinker. In other embodiments, adding riboflavin (i.e., vitamin B2) and an ammonium persulfate catalyst is required to activate the tyrosine residue crosslinker.

Other crosslinkers known in the art may be used as secondary crosslinkers in accordance with the compositions and methods described herein; as such, one of ordinary skill in the art would understand how to activate said crosslinkers.

In some embodiments, the streptavidin polypeptide forms a tetramer. In other embodiments, the streptavidin polypeptide forms a four-arm precursor. In some embodiments, the streptavidin polypeptide forms an eight-arm precursor. In further embodiments, the streptavidin polypeptide forms a twelve-arm precursor.

In some embodiments, the biopolymer composition is used for drug delivery. In other embodiments, the biopolymer composition is used as a nano or micron sized drug delivery system. In further embodiments, the biopolymer composition is used for imaging applications. In some embodiments, the drug is bound to the biopolymer composition described herein via biotin. In other embodiments, a growth factor may be bound to the biopolymer composition via biotin. In further embodiments, the biopolymer composition may be used for tissue engineering, including but not limited to vascular tissue (cardiac patch for the heart), blood vessels (arteries or veins), soft tissues (muscles or cartilage). In other embodiments, the biopolymer composition may be utilized in high temperature applications.

### Example

The following is a non-limiting example of the present invention. It is to be understood that said example is not intended to limit the present invention in any way. Equivalents or substitutes are within the scope of the present invention.

Interactions of self-oligomerizing biopolymers, such as proteins, play a crucial role in various biological processes, such as intercellular communication and the self-assembly of supramolecular structures (e.g., actin filaments, hemoglobin, virus capsid). Moreover, utilizing self-associating protein complexes as physical cross-linkers is advantageous for constructing intricate biomimetic materials requiring high specificity for biotechnological applications. However, the weak strength of such cross-linkers, due to physical interactions (i.e., hydrogen bonding, Van der Waals forces, hydrophobic interactions, electrostatic interactions), can lead to relatively fast erosion rates and, subsequently, in short material lifetimes. To enhance the strength of physically cross-linked materials, while maintaining high specificity, there is a need to develop a strategy that can modulate the mechanical strengths of protein complexes.

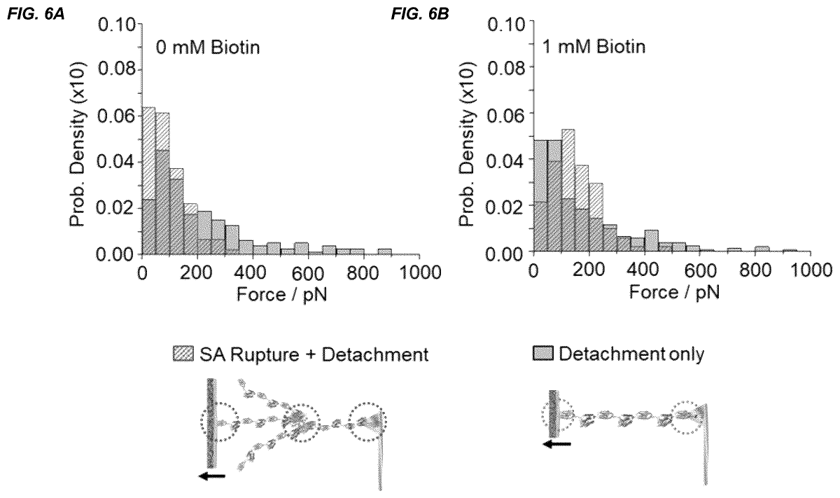

Reinforced weak dimer-dimer interface of SAv tetramer upon biotin binding: To evaluate the influence of biotin on the mechanical integrity of individual SAv tetramers, AFM was employed to measure the rupture strength of streptavidin (SAv) tetramers under different biotin conditions. For this purpose, a protein-based force probe composed of alternating tandem domains of I27 and staphylococcal nuclease (SNase) was genetically fused to the N-terminus of a SAv monomer (FIG. 5B). This force probe, consisting of mechanically strong (I27) and mechanically weak (SNase) domains, serves as a handle for AFM stretching measurements across three different directions (FIG. 5C). It acts as an internal force standard and provides an unmistakable fingerprint of true single molecule recordings. AFM studies revealed that at a pulling speed of 500 nm·s⁻¹, SNase and 127 domains unfold at approximately 30 pN with ˜45 nm of contour length increments (ΔLc) and 200 pN with ˜28 nm of ΔLc, respectively. The unique unfolding patterns of SNase and 127 served as an unambiguous detection of rupture forces (i.e., ≥30 pN) of individual ligand-receptor pairs and protein-protein complexes. Using these force probes, a molecular bridge can be easily formed through non-specific adsorption between an AFM tip and a substrate that can be stretched until the protein complex of interest ruptures and breaks apart (FIG. 5D). Because three SNase domains flank the protein complex, such as SAv tetramer, any force-extension recording containing four or more SNase domains indicates that the probes have been picked up on either side of the protein complex. The final force of such a recording can result from either the rupture strength of the protein complex, the force of detachment of the protein force probe from the substrate, or the AFM cantilever. To distinguish the rupturing forces of the protein complexes from the detachment events, the distribution of final force peaks of force probes was compared with and without the protein complex (FIG. 5E and FIGS. 6A and 6B).

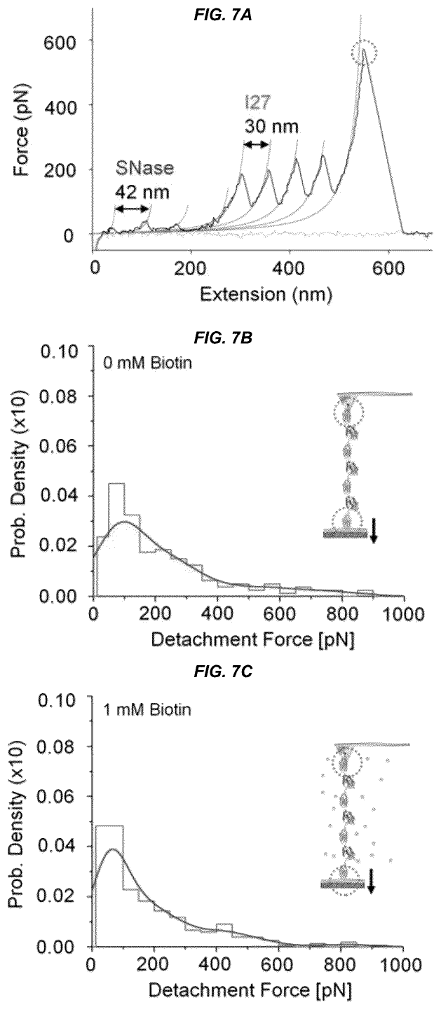

FIG. 5D presents a representative example of AFM force-extension curves of the single [(SNase-I27)₃-SAv monomer]₄ complex, which exhibited four or more SNase unfolding force peaks that fulfilled the criterion and were subsequently selected for further analysis. In the AFM force-extension curve, six characteristic weak unfolding force peaks of SNase domains were observed, indicating that the final force peak (dashed circle) represents either the rupture force of the SAv tetramer or the detachment force of I27-SNase handles from the substrate or the cantilever. To determine the rupture strength of the SA tetramer, the final force results of SAv tetramers with and without biotin were analyzed using kernel density estimation (KDE) and histogram methods (FIG. 5E and FIGS. 6A and 6B) and then compared with detachment force results of I27-SNase handles alone (FIGS. 6A and 6B). These probability densities showed that the most frequent rupture force of SAv tetramers without biotin occurs at around 50 pN (FIG. 5E; top). Comparing these final force events with detachment events at 50 pN allows the identification that more than 50% of final forces at 50 pN represent rupture events of SAv tetramers without biotin. In the presence of 1 mM biotin, biotin did not significantly affect detachment forces when compared to the result without biotin (FIGS. 7A, 7B, and 7C). However, the number of events with a final force at 50 pN was reduced. The center of the most frequent final force was shifted to 150 pN and had a greater proportion of events than the distribution of detachment events (FIG. 5E; bottom). This indicates that the interfaces within SAv tetramers strengthened from 50 pN to 150 pN when 1 mM biotin was present.

The most frequent final forces (FIG. 5E) mainly reflect rupture events of the SAv dimer-dimer interface. The SAv dimer-dimer interface has less interfacial surface area and less hydrogen bonding than the monomer-monomer interfaces based on the crystal structures. Additionally, the distributions are shown in FIG. 5E has long tails that extend up to 500 pN, whereas the distribution of detachment has events extending to 1000 pN. These results suggest that in FIG. 5E, 50 pN (top), and 150 pN (bottom) represent rupture events of the weak SA dimer-dimer interface, while the long tails in both distributions could be related to rupture events of strong monomer-monomer interfaces. The most frequent rupture force of the dimer-dimer interface at 100 pN is in the presence of 10 μM biotin. Therefore, biotin binding can enhance the mechanical strength of the dimer-dimer interface in individual SAv tetramers.

This finding can possibly be due to intersubunit contacts induced by tryptophan (W) 120. The contact between W120 and biotin may be attributed to the tighter association of the dimer-dimer interface. As a result, the enhancement of thermal stability of SAv tetramers has been suggested to be due to a tighter association of the dimer-dimer interface by the contact between W120 and biotin. Similar to the result of thermal stability, the mechanical stability of the dimer-dimer interface in the SAv tetramer is reinforced upon biotin binding (FIG. 5E).

Mechanical enhancement of SAv-crosslinked hydrogels upon biotin binding: Although the biotin binding to SAv tetramers enhanced their weak dimer-dimer interfacial strength at the single-molecule level, it remains unclear how this effect can be observed at the macroscale. To elucidate the macroscale implications of biotin binding, artificial protein hydrogels were designed and fabricated. These engineered hydrogels consist of a polymer network with interconnected SAv tetramers serving as crosslinking junctions. This approach enables effective mechanical characterization of the hydrogels, providing insights into the influence of biotin-mediated effects on bulk materials composed of SAv crosslinking junctions

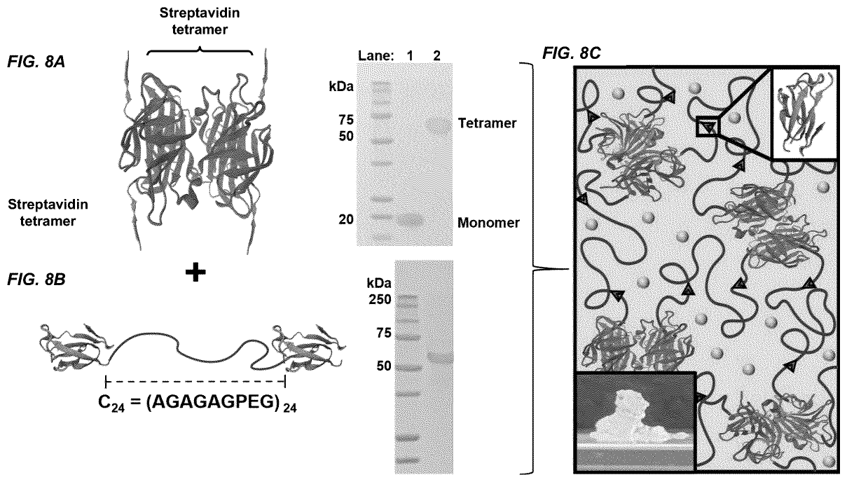

To promote the formation of a polymer network with SAv crosslinking junctions, an additional crosslinking mechanism with high specificity and strength is required. To ensure a cross-linked network, a “SpyTag-SpyCatcher” split protein system was utilized as the complementary crosslinking mechanism to the SAv tetramer cross-linker (FIGS. 8A, 8B, and 8C; Table 1). The key feature of this profound crosslinking mechanism involves the formation of an isopeptide bond between the Lys31 of SpyCatcher (SC) and Asp117 of SpyTag (ST) immediately after the self-association of these two biomacromolecules. Furthermore, two artificial protein constructs were biosynthesized. The first construct is a SAv tetramer with an ST genetically fused to the C-terminus of each SAv monomer, referred to as (SAv monomer-ST)₄ (FIG. 8A). The second construct, denoted as SC-C24-SC, is a well-known intrinsically disordered, flexible midblock protein composed of 24 repeats of the AGAGAGPEG (SEQ ID NO: 5) amino acid sequence, with an SC on each terminus (FIG. 8B). These protein solutions were mixed in 1:1 molar ratio between ST and SC, and the high specificity of both crosslinking mechanisms enabled the protein network to be self-assembled and form a hydrogel (FIG. 8C) without the mechanisms interfering with each other.

Table 1: List of theoretical molecular weight (M.W.) and liquid chromatography-mass spectrometry (LC/MS) M.W. of the protein constructs post synthesis. For each sample, the recorded M.W. with the highest relative intensity is listed in the table below.

| Protein Construct | Theoretical MW (kDa) | LC/MS MW (kDa) |
|---|---|---|
| (SAv monomer-ST)₁ | 16.52 | 16.52 |
| SC—C₂₄—SC | 41.60 | 41.66 |
| SC—NI₆C—SC | 49.36 | 49.37 |

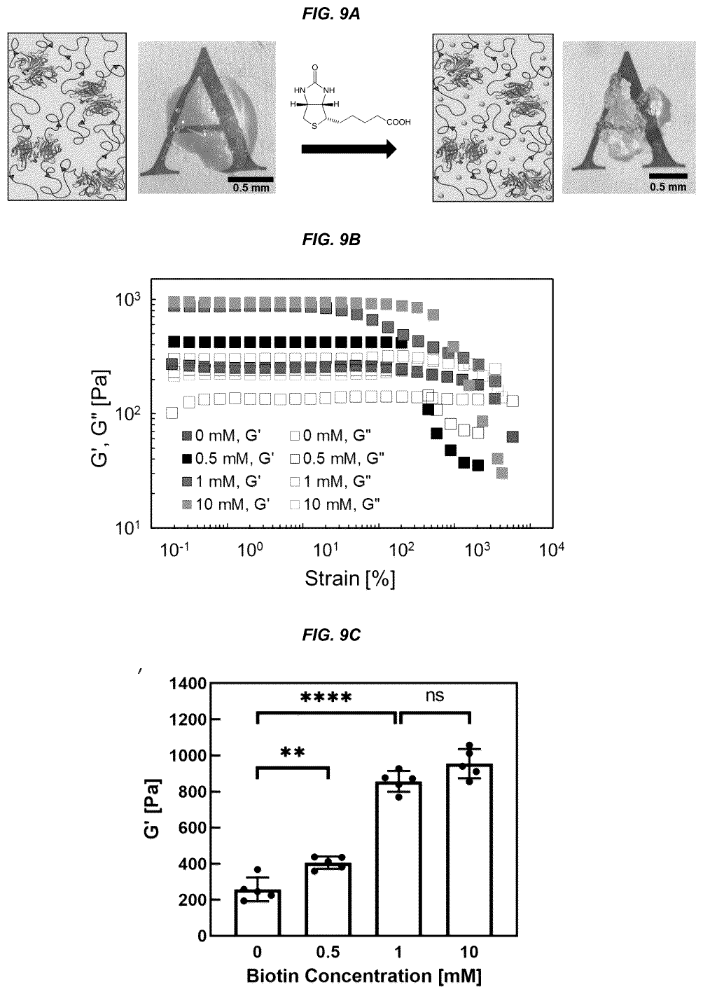

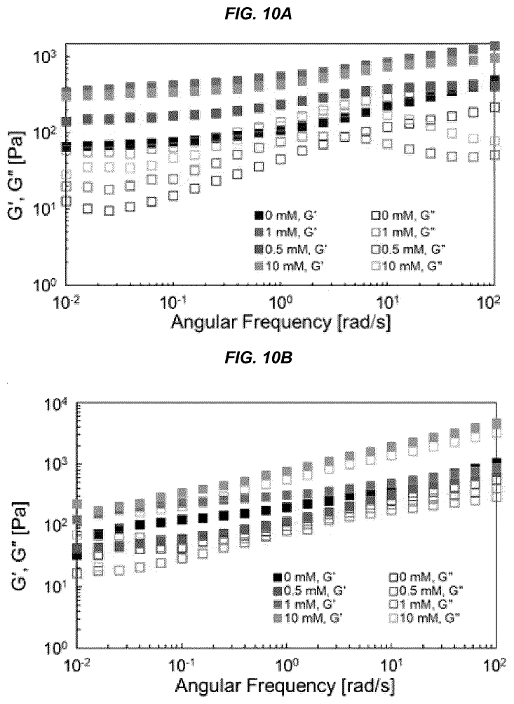

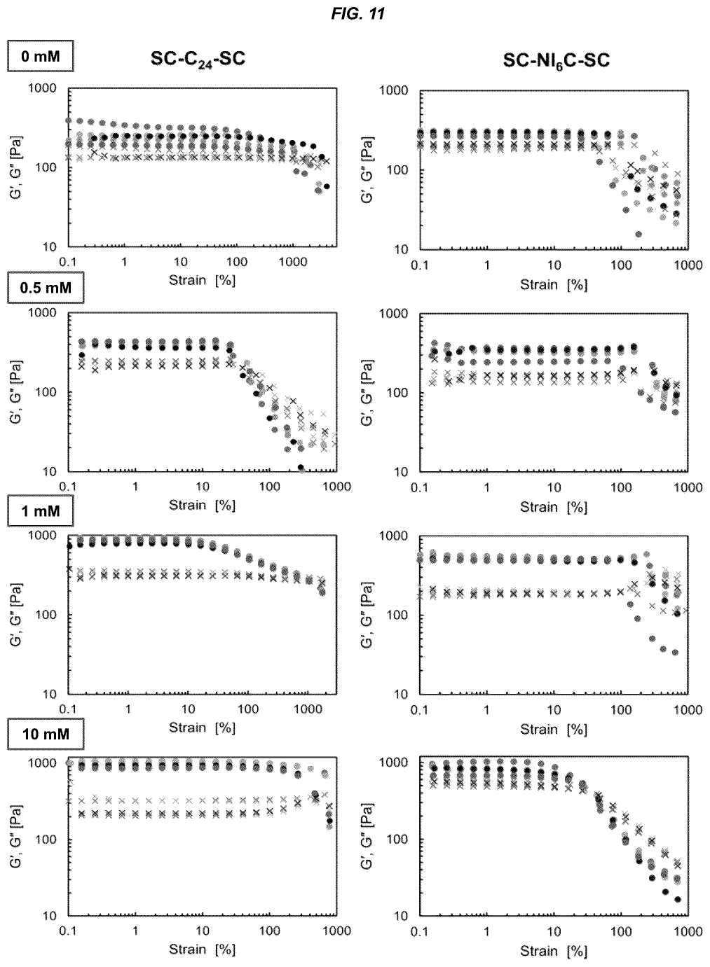

By visual inspection, the addition of biotin caused the hydrogels to be compact (FIG. 9A). To quantify this unprecedented finding, the mechanical strength of SAv-based hydrogels was evaluated at different concentrations of biotin using small amplitude oscillatory shear rheology. The shear elastic modulus (G′) through strain was compared to the frequency sweep measurements (FIG. 9B, FIGS. 10A and 10B, and FIG. 11). The results indicate that biotin binding to SAv tetramers significantly improves the experimental G′ of the hydrogel (FIG. 9C; Table 2). When examining the experimental G′ at 0.5 mM and 1 mM biotin concentrations, values of 339.81±18.31 Pa and 856.83±58.20 Pa were observed, respectively. These data reveal a 32% increase in the experimental G′ at 0.5 mM and a dramatic 232% increase at 1 mM, relative to the baseline value of 257.85±65.94 Pa at 0 mM biotin. Based on the substantial improvements in G′ at 1 mM of biotin, the biotin concentration was further increased to 10 mM. The hydrogel, containing approximately 3.6 mM of SAv tetramer, exhibited a slight increase in the experimental G′ to 954.79±80.72 Pa compared to the 1 mM biotin concentration. Although this difference was statistically significant, it was evident that increasing the biotin concentration substantially influenced the shear elasticity of the SAv-based hydrogels.

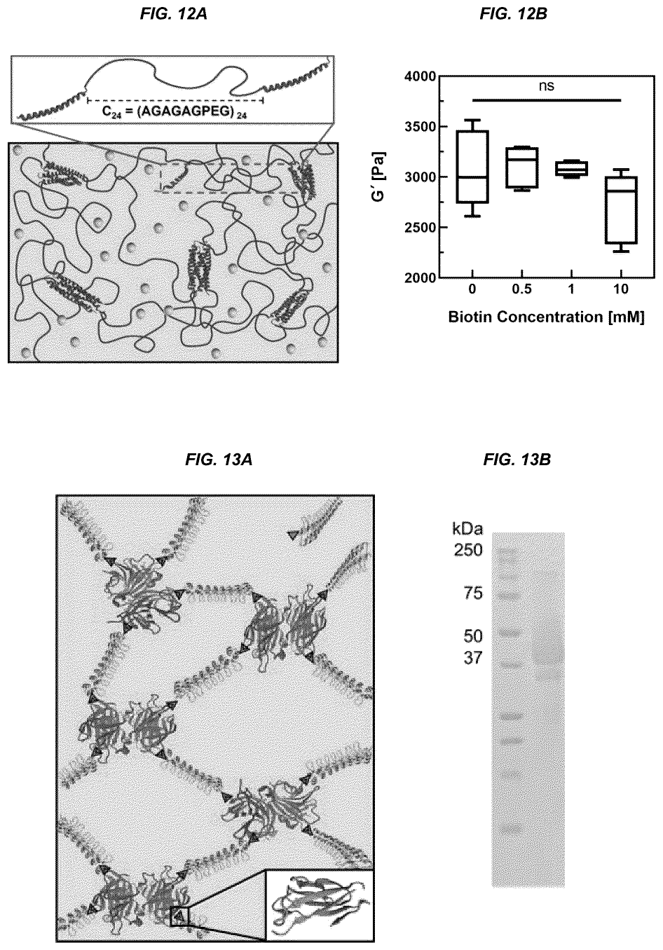

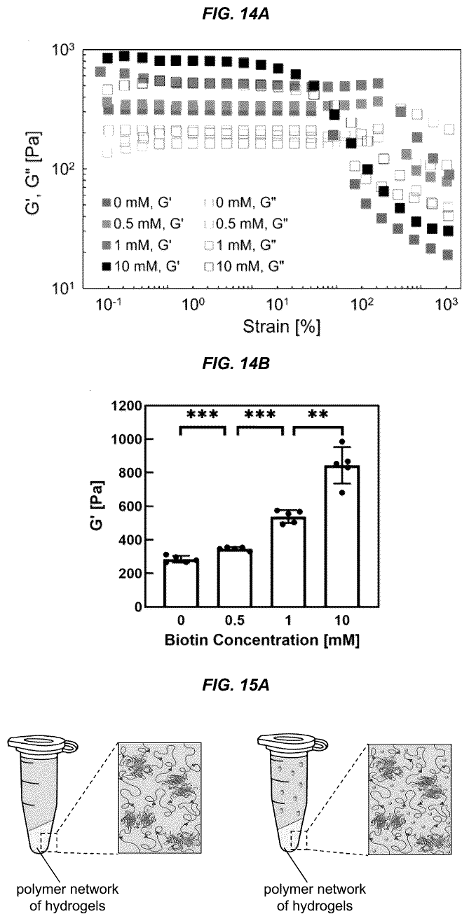

However, to affirm that these results were exclusively due to the binding of biotin to SAv tetramers, additional control experiments were required. In these experiments, hydrogels were fabricated and characterized where the SAv crosslinking junctions were replaced with pentameric complexes composed of self-assembling physical coiled-coil cross-linkers (FIGS. 12A and 12B). There were no significant changes in experimental G′ regardless of the biotin concentration (FIG. 12B). Furthermore, another control was designed using a different SAv-based protein polymer design, where the flexible midblock was replaced with a more rigid structure (SC-NI₆C-SC) using a synthetic version of ankyrin, a spring-like protein found in the cytoskeleton of red blood cells (FIGS. 13A and 13B). Despite changing the strand rigidity, there was still a significant increase in experimental G′ across different biotin concentrations (FIGS. 14A and 14B and Table 2). As a result, these additional controls validate the experimental results (FIGS. 9A, 9B, and 9C) and support the notion that the biotin binding on SAv crosslinking junctions can enhance the overall mechanical properties of these hydrogels.

TABLE 2
Significant differences in experimental shear G′ across various
biotin concentrations. Each dataset (row) was compared to the
dataset containing 0 mM biotin using two-sided, independent t-
tests with Welch's correction (see Experimental Methods below).

| | | SAv Hydrogel w/ | | SAv Hydrogel w/ | |
|---|---|---|---|---|---|
| Biotin | | SC—C₂₄—SC | | SC—NI₆C—SC | |
| Concentration | | (N = 5) | | (N = 5) | |
| 0 | mM | 257.85 ± 65.94 | Pa | 284.12 ± 20.11 | Pa |
| 0.5 | mM | 339.81 ± 18.31 | Pa * | 346.36 ± 10.15 | Pa *** |
| 1 | mM | 856.83 ± 58.20 | Pa **** | 538.65 ± 38.05 | Pa **** |
| 10 | mM | 954.79 ± 80.72 | Pa **** | 843.99 ± 108.67 | Pa *** |

* p < 0.05;
**p < 0.01;
*** p < 0.0005;
**** p < 0.0001

Given the methodological disparity in techniques between AFM-based SMFS and rheology, it is difficult to directly correlate the mechanical reinforcements of individual SAv tetramers with the observed improvements in shear elasticity of SAv-based hydrogels. Biotin binding to the SAv tetramer induces a tightening effect on the SAv tetramer, causing a twisting motion at the dimer-dimer interface. Thus, the collective change in every individual SAv structure significantly contributes to the mechanical enhancement of the overall hydrogel comprised of crosslinked SAv tetramers. With approximately 49% of the polymer network consisting of SAv tetramers, it is plausible that nearly half of the polymer network may experience structural changes. This, in turn, could lead to increased stress in the strands between SAv-crosslinkers within the polymer network upon biotin binding. Therefore, this conjecture, coupled with the visual inspection in FIG. 9A suggests an indirect correlation between nanoscale and macroscale findings.

A method to directly compare macroscale and nanoscale results is to conduct tensile testing on bulk SAv-based materials at different biotin concentrations due to the similar methodology employed in AFM-based SMFS. However, this SAv-based polymer network design, despite demonstrating mechanical enhancement of SAv tetramers in the presence of biotin, exhibited limitations in overall strength. It is not sufficiently strong enough for tensile testing primarily due to the presence of network defects. These defects, specifically inhomogeneous crosslinking density, can weaken the overall material enough to limit mechanical characterization. This deficiency becomes apparent when examining the experimental G′ at a 10 mM biotin concentration, which was only 15.97±1.35% of the theoretical G′ calculated by the Affine network model (Table 3). The major cause of this inhomogeneity can be attributed to the uncontrollable, spontaneous chemical crosslinking after the specific molecular self-assembly between the ST and SC. The rapid isopeptide bond formation does not provide enough time for adequate diffusion of the SAv tetramers throughout the polymer network. Considering these issues and the current lack of a more effective crosslinking mechanism that offers temporal control without interfering with SAv tetramer self-assembly, the ST-SC system was selected as the suitable option. For this reason, it is evident that further optimization to the SAv-based polymer network design is needed, or a different type of macroscale experiment is required to directly correlate nanoscale results to the macroscale level.

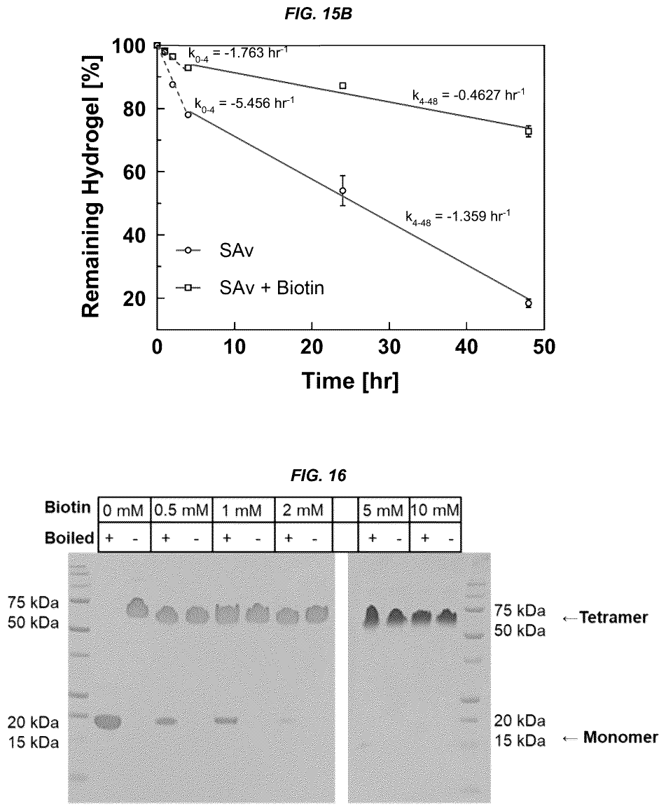

TABLE 3
Predictions of the theoretical shear G′ were made using Affine and
Phantom network models. The methods to calculate these predictions are known in the art.

| SAv Cross-linker | Molecular weight of protein | | Phantom | *Affine G′ |
|---|---|---|---|---|
| functionality | between SAv cross-linkers | v (m⁻³) | G′ (kPa) | (kPa) |
| 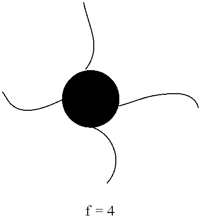 | 74.60 kDa; SA-C₂₄-SA (FIG. 15A and 15B) 82.36 kDa; SAv-Nl₆C-SAv (FIG. 13A and 13B) | 1.45 × 10¹⁷ 1.32 × 10¹⁷ | 2.99 2.71 | 5.98 5.42 |

Improvements in bioerosion rates in SAv-based hydrogels upon biotin binding: To explore the impact of biotin-mediated mechanical reinforcements of SAv tetramers in bulk materials, an alternative approach was taken and bioerosion experiments were conducted. Bioerosion, resulting in the loss of mechanical strength and structure due to the breakdown of cross-linked sites in a polymer network, was considered a suitable method to directly examine whether biotin binding can reinforce and strengthen SAv-based hydrogels at the macroscale. In this investigation, the erosion rate was evaluated of SAv-based hydrogels with and without biotin under erosion-promoting conditions at 37° C. (FIG. 15A). Based on the rheological characterization (FIG. 9C) and SDS-PAGE analysis (FIG. 16), the highest shear elasticity and the preservation of every individual SAv tetramers within the polymer network under denaturing conditions were achieved at a biotin concentration of 10 mM, respectively. This confirms that 10 mM biotin reinforces every SAv tetramer in the polymer network, making it a reasonable concentration to test.

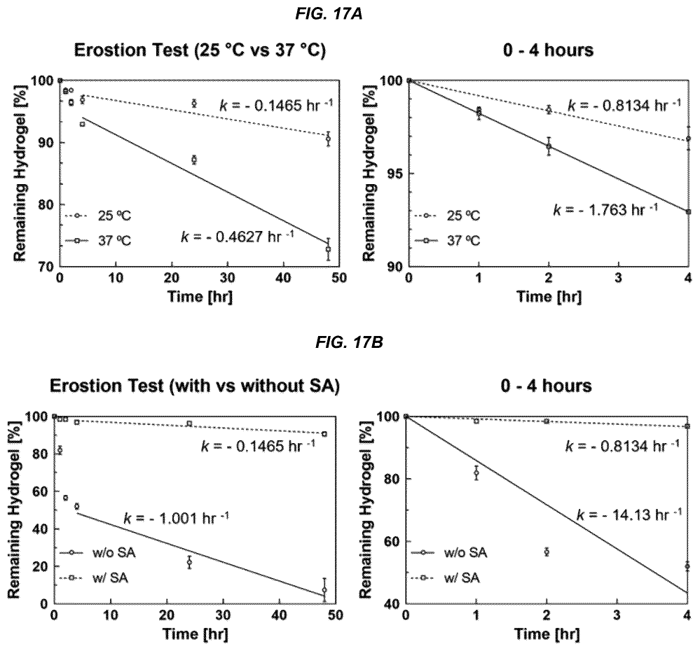

Furthermore, a comparative study was conducted using hydrogels with physical coiled-coil cross-linkers as a reference, in contrast to hydrogels with SAv cross-linkers (FIGS. 17A and 17B). This comparison revealed that SAv tetramers without biotin can function as strong physical cross-linkers compared to other types. This provides perspective into the inherent strength of SAv tetramer without biotin as a physical cross-linker, which can be enhanced in the presence of biotin.

To gain insight into the effects of how biotin binding affects the overall erosion dynamics, the erosion rates of SAv-based hydrogels were examined with or without 10 mM biotin. The analysis revealed distinct erosion rates during two specific timeframes: 0-4 hours and 4-48 hours (FIG. 15B). Surprisingly, regardless of the presence of biotin, all hydrogels exhibited a significant increase in the absolute erosion rate within the initial 0-4 hours, ranging approximately 280-300%, compared to the subsequent 4-48 hours. This rapid erosion rate during the initial stages may be attributed to the presence of non-crosslinked proteins within the polymer network, increasing the likelihood of detaching from the hydrogel surface. To ensure a more accurate assessment of the erosion process, the analysis focused on the erosion rate between 4 and 48 hours. This specific timeframe was chosen because it represents a period where significant differences in erosion rates were observed. At this stage, it is expected that the majority of non-crosslinked proteins have been detached, and the hydrogel stability has become more established.

Within the time frame of 4-48 hours, we observed a 66% decrease in erosion rate in the hydrogels with mechanically reinforced SAv tetramers when compared to the hydrogels without biotin in the buffer (FIG. 15B). Using these erosion rates, the half-life of the hydrogels was calculated with mechanically reinforced SAv tetramers to be approximately 100 hours, while the half-life of the hydrogels without biotin was only about 25 hours. This substantial 300% increase in the hydrogel half-life indicated that ligand binding mediates the mechanical enhancements of the individual SAv tetramers, reinforcing the crosslinking sites in the polymer network and effectively delaying erosion over time. Therefore, these findings established a direct correlation between the nanoscale mechanical reinforcement observed in AFM-based SMFS studies and the improved erosion resistance at the macroscale, emphasizing the critical role of ligand-mediated mechanical reinforcement in enhancing the overall stability of the hydrogels across multiple scales.

The present invention features SAv tetramers genetically incorporated into artificial protein polymer network designs and fabricated SAv-based hydrogels. Additionally, described herein is the mechanical impact of biotin binding at the macroscale. Not only did these hydrogels demonstrate significant enhancements in mechanical strength in the polymer network, caused by the biotin binding effects to the SAv cross-linkers, but they also exhibited a substantial decrease in erosion rates, indicating improvements in the material lifespan. By demonstrating that biotin binding effects at the nanoscale can influence the overall polymer network composed of the SAv tetramer-based cross-linkers, thus, the present invention features a simplistic approach to mechanically tune not only self-assembled protein complexes but also more sophisticated architectures with a prolonged lifespan.

Experimental Methods

Sample preparation for AFM-based single-molecule force microscopy: [(I27-SNase)₃-SAv monomer]₄ and I27-(SNase-I27)₃ constructs were prepared by the method as described previously.³² 1 mg/mL of all proteins were diluted to 10-100 μg/mL by the buffer containing 100 mM Tris-HCl (pH 7.6), 150 mM NaCl, 1 mM EDTA, and 1 mM Tcep with or without 1 mM biotin (product #B4501, Sigma-Aldrich, St. Louis, MO, USA). Fresh Au coverslips were prepared right before the protein incubation on those substrates in order to provide similar surface conditions. Au-coated glass coverslips were sonicated in acetone and then ethanol for 3 minutes each. [(I27-SNase)₃-SAv monomer]₄ and I27-(SNase-I27)₃ proteins were incubated on fresh Au substrates. 50 μL of protein solutions with 5˜ 20 μg/mL concentration were used for all samples. After 40 minutes of incubation at room temperature, all proteins were gently washed by the buffer and ready for AFM pulling experiments.

AFM-based single-molecule force spectroscopy measurements: Measurements of rupture forces or detachment forces of protein complexes were performed by our custom-built AFM instruments with MSNL AFM cantilevers (Bruker AFM Probes, Santa Barbara, CA, USA). 10˜18 pN/nm of AFM cantilever spring constants were obtained in solution at room temperature by using the energy equipartition theorem. The cantilever produced ˜10 pN of RMS force noise in the 1-500 Hz bandwidth. To avoid obtaining final force results from the same molecule, the AFM tip only pulled molecules once at each location. An AFM automation automatically adjusted the Z-axis to perform force extension experiments while the X-Y locations were raster scanned across the surface. Usable force-extension curves were recorded automatically if they fulfilled heuristics present in “good” recordings.

Protein expression for SA-based hydrogel characterization: Amino acid sequences for (SAv monomer-ST)₄, SC-C₂₄-SC, SC-NI₆C-SC, and P-C₂₄-P constructs were recorded in Table 4. Gene sequences were inserted into the pET24a expression plasmids using BamHI, XhoI restriction enzymes (GenScript, USA). The resulting plasmids were then transformed into BL21(DE3) Escherichia coli(New England Biolabs, USA) and grown on kanamycin-resistant agar plates overnight. Single colonies were used to inoculate 5 mL of TB medium with 50 μg/L kanamycin, which was then grown in a shaking incubator at 37° C. and 220 RPM overnight. Then, 10 mL of the overnight culture was added to a 2.8 L protein expression flask containing 900 mL TB media and 100 mL of potassium phosphate buffer with 1 mL of 50 mg/L kanamycin. These 1 L cultures were incubated at 37° and 250 RPM until the optical density at a wavelength of 600 nm (OD₆₀₀) was between 0.5 to 0.7, which was measured using a Cary 60 UV-vis spectrophotometer (Agilent Technologies, USA). Once the OD₆₀₀ reached the absorbance range, 500 μL of 1 M isopropyl β-D-1-thiogalactopyranoside (IPTG) was added to every 1 L culture, which induces protein expression, and the cultures were incubated overnight at 24° C. and 220 RPM. Using centrifugation, cells were harvested at 4° C. at 7,000×g for 10 minutes. Cell pellets from each 1 L expression were first suspended in 200 mL of 50 mM sodium phosphate and 300 mM sodium chloride (pH 7.6) and then frozen at −80° C. for at least 1 hour. For (SAv monomer-ST)₄ expression, cell pellets must be resuspended in the same buffer with 8 M Urea to prevent any formation of inclusion bodies.

TABLE 4
Full amino acid sequences for each construct used for bulk material
characterization studies on SAv-based hydrogels. All constructs have a protein yield of ~20-30
mg per L culture. SAv monomer cross-linker (italicized), P coiled-coil cross-linkers (bolded), C₂₄
(underlined gray highlight), NI₆C (italicized and bolded), SpyTag (ST; italicized and underlined),
SpyCatcher (SC; bolded and underlined).

| | | SEQ |
|---|---|---|
| Protein Name | Protein Sequence | ID NO: |
| SAv monomer- | MHHHHHHGSGGSAEAGITGTWYNQLGSTFIVTAGADGALTGTY | 1 |
| ST | ESAVGNAESRYVLTGRYDSAPATDGSGTALGWTVAWKNNYRN | |
| | AHSATTWSGQYVGGAEARINTQWLLTSGTTEANAWKSTLVGH | |
| | DTFTKVKPSAASASGSTSAHIVMVDAYKPTK | |
| SC-C₂₄-SC | MHHHHHHGSGGSDSATHIKFSKRDEDGKELAGATMELRDSSG | 2 |
| | KTISTWISDGQVKDFYLYPGKYTFVETAAPDGYEVATAITFTVN | |
| | EQGQVTVNGKATKGDAHIASYRDPMGAGAGAGPEGAGAGAG | |
| | PEGAGAGAGPEGAGAGAGPEGAGAGAGPEGAGAGAGPEGAG | |
| | AGAGPEGAGAGAGPEGAGAGAGPEGAGAGAGPEGARMPTSY | |
| | RDPMGAGAGAGPEGAGAGAGPEGAGAGAGPEGAGAGAGPE | |
| | GAGAGAGPEGAGAGAGPEGAGAGAGPEGAGAGAGPEGAGAG | |
| | AGPEGAGAGAGPEGARMPTSYRDPMGAGAGAGPEGAGAGAG | |
| | PEGAGAGAGPEGAGAGAGPEGARMPEFDSATHIKFSKRDEDG | |
| | KELAGATMELRDSSGKTISTWISDGQVKDFYLYPGKYTFVETA | |
| | APDGYEVATAITFTVNEQGQVTVNGKATKGDAHI | |
| SC-NI₆C-SC | MHHHHHHGSGGSDSATHIKFSKRDEDGKELAGATMELRDSSG | 3 |
| | KTISTWISDGQVKDFYLYPGKYTFVETAAPDGYEVATAITFTVN | |
| | EQGQVTVNGKATKGDAHIASGSGT [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] TSGSEFDSATHIKFSKRDEDGKEL | |
| | AGATMELRDSSGKTISTWISDGQVKDFYLYPGKYTFVETAAPD | |
| | GYEVATAITFTVNEQGQVTVNGKATKGDAHI | |
| P-C₂₄- P² | MGSGSGSAPQMLRELQETNAALQDVRELLRQQVKEITFLKNT | 4 |
| | VMESDASGASYRDPMGAGAGAGPEGAGAGAGPEGAGAGAGP | |
| | EGAGAGAGPEGAGAGAGPEGAGAGAGPEGAGAGAGPEGAGA | |
| | GAGPEGAGAGAGPEGAGAGAGPEGARMPTSYRDPMGAGAGA | |
| | GPEGAGAGAGPEGAGAGAGPEGAGAGAGPEGAGAGAGPEGA | |
| | GAGAGPEGAGAGAGPEGAGAGAGPEGAGAGAGPEGAGAGAG | |
| | PEGARMPTSYRDPMGAGAGAGPEGAGAGAGPEGAGAGAGPE | |
| | GAGAGAGPEGARMPEFAPQMLRELQETNAALQDVRELLRQQ | |
| | VKEITFLKNTVMESDASGKLAAALEHHHHHH | |

Protein purification for SA-based hydrogel characterization: Frozen, resuspended cell pellets were thawed and transferred into 500 mL wide-mouth bottles. The resuspended samples were sonicated three times for ten minutes each using a Branson Sonifier 250 (Branson Ultrasonics, Danbury, CT, USA) at an output power of 5 and a duty cycle of 50%. Once the sample viscosity resembles the viscosity of water, the solution was poured into 500 mL centrifuge bottles and centrifuged at 20,000×g for 40 minutes at 10° C. To further remove any more insoluble cell debris, the supernatant was aliquoted into 50 mL conical tubes and then centrifuged at 12,500×g for 20-30 minutes at 10° C. Then, the supernatant was stored at 4° C. Due to the six Histidine amino acid residues on one of the termini, the desired proteins were purified and extracted via affinity column chromatography using Cobalt-Resin beads (Fisher Sci, Catalog: P189965, USA). Column elutions were dialyzed in 4.5 L of 20 mM Tris buffer (pH 8.0), with a total of seven buffer changes occurring every three hours to remove any salts, impurities, and/or urea. Following tris dialysis, fast protein liquid chromatography was performed in a 20 mM Tris buffer (pH 8.0) with an anion exchange column. Selected elutions were collected and dialyzed in 4.5 L deionized water following the same protocol as the Tris dialysis step to remove any impurities. After the entire purification procedure, all purified proteins were lyophilized and stored at −20° C. Sample purity was evaluated using SDS-PAGE analysis after each purification step and using LC/MS after biosynthesis (see Table 1).

SA-based hydrogel fabrication: All lyophilized protein constructs were dissolved in 20 mM Tris at 4° C. overnight. For the stock (SAv monomer-ST)₄ solution, biotin was added and dissolved to fulfill the desired final biotin concentration. Stock concentration of (SAv monomer-ST)₄ and SC-C₂₄-SC, or SC-NI₆C-SC, were mixed where the final concentration has a 1:1 molar ratio between ST and SC. Signs of gelation occurred within 5 minutes. To make sure all the possible self-assembly between ST and SC is completed, hydrogel samples were further incubated in 4° C. for 1 hour right before characterization.

Rheology: Mechanical properties of each hydrogel were characterized by small amplitude oscillatory shear rheology using the Discovery Hybrid Rheometer 2 (TA Instruments, New Castle, DE, USA) with a sandblasted 1°, 20 mm cone-plate geometry. Before each experiment, inertia and friction of the geometry and rotational mapping calibrations were performed. All experiments were maintained at 25° C. using a Peltier temperature-controlled stage. To prevent evaporation, mineral oil was spread around the stage, and a solvent trap with a DI H₂O seal was placed over the geometry. Strain sweeps were conducted from 0.1-1000% shear strain at a constant angular frequency of 10 rad s⁻¹. G′ and G″ were calculated by averaging the values within the linear viscoelastic region (0.1-1%). Frequency sweeps were conducted from 0.01 to 100 rad s⁻¹ with a constant shear strain of 1%, all within the linear viscoelastic region.

Statistical data analysis: Rheology datasets were analyzed using a two-sided, independent sample t-test to compare the experimental shear elastic modulus (G′) of the hydrogel samples with varying biotin concentrations. The sample size (N) for all experiments and p-values for all statistical analyses were reported in Table 2. All experimental values were written as the mean±standard deviation in Table 2 and throughout the manuscript unless otherwise noted. All analysis was conducted using Microsoft Excel and GraphPad Prism 9. For post-processing, any data that were over 2 standard deviations of the mean were considered as outliers and were removed.

Theoretical network model calculations: According to methods in the referenced articles, theoretical shear G′ was calculated using affine and phantom polymer network models (Table 3).

Bioerosion test: 20 μL of hydrogels with either 0 or 10 mM biotin were centrifuged in 1.5 mL microcentrifuge tubes to ensure that all the sample reached the bottom. Then, 180 μL of 20 mM Tris buffer (pH 8.0) with or without 10 mM biotin were gently pipetted in each microcentrifuge tube. All samples were incubated at 37° C. without any mechanical agitation. To reduce the evaporation of the buffer, each tube was wrapped with parafilm. At each time point, the absorbance of 1 μL of the centrifuged supernatant was measured using a Nanophotometer P-330 and a submicroliter cell with a pathlength of 1 mm (Implen, Westlake Village, CA, USA). The sample size of each time point was in triplicate (N=3), with three separate hydrogel samples measured.

### EMBODIMENTS

The following embodiments are intended to be illustrative only and not to be limiting in any way:

### Embodiment A

Embodiment 1A: A polypeptide monomer composition effective as a nano spring, the composition comprising: a first crosslinker linked to an ankyrin component comprising a plurality of ankyrin repeats linearly connected.

Embodiment 2A: The composition of embodiment 1A, wherein the first crosslinker comprises a streptavidin polypeptide, an avidin polypeptide, or a derivative thereof.

Embodiment 3A: The composition of embodiment 1A or embodiment 2A, further comprising a secondary crosslinker linked to the ankyrin component by a linker.

Embodiment 4A: The composition of embodiment 3A, wherein the secondary crosslinker is a chemical crosslinker.

Embodiment 5A: The composition of embodiment 4A, wherein the chemical crosslinker is GB1.

Embodiment 6A: The composition of embodiment 4A, wherein the chemical crosslinker is a photocrosslinker.

Embodiment 7A: The composition of embodiment 3A, wherein the secondary crosslinker is a physical crosslinker.

Embodiment 8A: The composition of any one of embodiments 1A-7A, further comprising a biotin molecule bound to the first crosslinker.

Embodiment 9A: The composition of embodiment 8A, wherein the biotin molecule further comprises a secondary crosslinker linked by a linker.

Embodiment 10A: The composition of any one of embodiments 3A-9A, wherein the linker is a protein based linker.

Embodiment 11A: The composition of embodiment 10A, wherein the protein based linker is a structured protein.

Embodiment 12A: The composition of any one of embodiments 3A-11A, wherein the linker is a flexible linker.

Embodiment 13A: The composition of any one of embodiments 1A-12A, further comprising a biotinylated protein bound to the first crosslinker.

Embodiment 14A: The composition of any one of embodiments 1A-13A, wherein the ankyrin component comprises about 3 to 24 ankyrin repeats.

Embodiment 15A: A biopolymer composition comprising a plurality of polypeptide monomer compositions according to any one of embodiments 1A-14A linked together.

Embodiment 16A: The composition of embodiment 15A, the biopolymer composition promotes ankyrin repeat protein mechanics.

Embodiment 17A: The composition of embodiment 15A or embodiment 16A, the biopolymer composition promotes nanospring mechanics.

Embodiment 18A: The composition of any one of embodiments 15A-17A, wherein the biopolymer composition is used for drug delivery

Embodiment 19A: A method of producing a biopolymer composition, the method comprising: a) solubilizing a plurality of polypeptide monomers, each monomer comprising: i) a first crosslinker; ii) an ankyrin component comprising a plurality of ankyrin repeats linearly connected together linked to the first crosslinker; and iii) a secondary crosslinker linked to the ankyrin component by a linker; wherein solubilizing the polypeptide monomers allows for the first crosslinkers to associate together; and b) activating the secondary crosslinkers such that the secondary crosslinkers associate together.

Embodiment 20A: The method of embodiment 19A, wherein the first crosslinker comprises a streptavidin polypeptide, an avidin polypeptide, or a derivative thereof.

Embodiment 21A: The method of embodiment 19A or embodiment 20A further comprising adding biotin before activating the secondary crosslinkers, wherein biotin binds to the first crosslinker.

Embodiment 22A: The method of embodiment 19A or embodiment 20A further comprising adding biotin after activating the secondary crosslinkers, wherein biotin binds to the first crosslinker.

Embodiment 23A: The method of embodiment 19A or embodiment 20A further comprising adding a biotinylated protein before activating the secondary crosslinkers, wherein the biotinylated protein binds to the first crosslinker.

Embodiment 24A: The method of embodiment 19A or embodiment 20A further comprising adding a biotinylated protein after activating the secondary crosslinkers, wherein the biotinylated protein binds to the first crosslinker.

Embodiment 25A: The method of any one of embodiments 19A-24A, wherein the secondary crosslinker is a chemical crosslinker.

Embodiment 26A: The method of embodiment 25A, wherein the secondary crosslinker is GB1.

Embodiment 27A: The method of embodiment 26A, wherein GB1 is activated by the removal of (dithiothreitol) DTT.

Embodiment 28A: The method of any one of embodiments 19A-24A, wherein the secondary crosslinker is a physical crosslinker.

Embodiment 29A: The method of any one of embodiments 19A-24A, wherein the secondary crosslinker is a photocrosslinker.

Embodiment 30A: The method of embodiment 29A, wherein the photocrosslinker comprises tyrosine residue crosslinker.

Embodiment 31A: The method of embodiment 30A, wherein the tyrosine residue crosslinker is activated by adding a ruthenium and an ammonium persulfate catalyst and exposing said crosslinker to a light.

Embodiment 32A: The method of embodiment 30A, wherein the tyrosine residue crosslinker is activated by adding a riboflavin and an ammonium persulfate catalyst and exposing said crosslinker to a light.

Embodiment 33A: The method of embodiment 30A or 31A, wherein the light is a blue light.

Embodiment 34A: The method of embodiment 30A or 31A, wherein the light is UV light.

Embodiment 35A: The method of embodiment 19A, wherein the linker is flexible.

Embodiment 36A: The method of embodiment 19A, wherein the linker is protein based.

Embodiment 37A: The method of embodiment 19A, wherein the ankyrin component comprises 3 to 24 ankyrin repeats.

Embodiment 38A: The method of embodiment 19A, wherein streptavidin polypeptide forms a tetramer.

Embodiment 39A: The method of embodiment 19A, wherein streptavidin polypeptide forms a four-arm precursor.

Embodiment 40A: The method of embodiment 19A, wherein the streptavidin polypeptide forms an eight-arm precursor.

Embodiment 41A: The method of embodiment 19A, wherein the streptavidin polypeptide forms up to a twelve-arm precursor.

Embodiment 42A: A biopolymer composition comprising a plurality of polypeptide monomers linked together, each polypeptide monomer comprising: a) a first crosslinker; b) an ankyrin component comprising a plurality of ankyrin repeats linearly connected together linked to the first crosslinker; and c) a secondary crosslinker linked to the ankyrin component by a flexible linker, wherein the streptavidin polypeptides associate together, and wherein the secondary crosslinkers associate together to generate the biopolymer composition.

Embodiment 43A: The composition of embodiment 42A, wherein the first crosslinker comprises a streptavidin polypeptide, an avidin polypeptide, or a derivative thereof.

### Embodiment B

Embodiment 1B: A method of modulating mechanical properties of a biopolymer composition, the method comprising: a) solubilizing a plurality of polypeptide monomers, each monomer comprising: i) a first crosslinker; and ii) a repeat component comprising a plurality of amino acid repeats linearly connected together linked to the first crosslinker; and b) adding a concentration of biotin or a derivative thereof to the solubilized polypeptide monomers; wherein solubilizing the polypeptide monomers allows for the first crosslinkers to associate together to form the biopolymer composition; and wherein the concentration of biotin modulates the mechanical properties of the biopolymer composition.

Embodiment 2B: The method of embodiment 1B, wherein the first crosslinker comprises a streptavidin polypeptide, an avidin polypeptide, or a derivative thereof.

Embodiment 3B: The method of embodiment 1B or embodiment 2B, wherein the repeat component comprises an ankyrin component comprising a plurality of ankyrin repeats linearly connected together linked to the first crosslinker.

Embodiment 4B: The composition of embodiment 3B, wherein the ankyrin component comprises 3 to 24 ankyrin repeats.

Embodiment 5B: The method of embodiment 1B, wherein the repeat component comprises SEQ ID NO: 6 or SEQ ID NO: 7.

Embodiment 6B: The method of any one of embodiments 1B-5B, wherein each monomer further comprises a secondary crosslinker linked to the repeat component by a linker.

Embodiment 7B: The method of embodiment 6B, wherein the secondary crosslinker is a chemical crosslinker.

Embodiment 8B: The method of embodiment 7B, wherein the secondary crosslinker comprises either a GB1A or GB1B

Embodiment 9B: The method of embodiment 7B, wherein the secondary crosslinker comprises either a SpyCatcher or SpyTag.

Embodiment 10B: The method of any one of embodiments 1B-9B, wherein the concentration of biotin or a derivative thereof is about 0.01 mM to 10 mM.

Embodiment 11B: A polypeptide monomer composition effective as a nano spring, the composition comprising: a first crosslinker linked to a repeat component comprising a plurality of amino acid repeats linearly connected.

Embodiment 12B: The composition of embodiment 11B further comprising biotin or a derivative thereof bound to the receptor of the first crosslinker.

Embodiment 13B: A polypeptide monomer composition effective as a nano spring, the composition comprising: a first crosslinker linked to a repeat component comprising a plurality of amino acid repeats linearly connected and biotin or a derivative thereof bound to the receptor of the first crosslinker.

Embodiment 14B: The composition of any one of embodiments 11B-13B, wherein the first crosslinker comprises a streptavidin polypeptide, an avidin polypeptide, or a derivative thereof.

Embodiment 15B: The composition of any one of embodiments 11B-14B, wherein the repeat component comprises an ankyrin component comprising a plurality of ankyrin repeats linearly connected together linked to the first crosslinker.

Embodiment 16B: The composition of embodiment 15B, wherein the ankyrin component comprises 3 to 24 ankyrin repeats.

Embodiment 17B: The composition of any one of embodiments 11B-14B, wherein the repeat component comprises SEQ ID NO: 6 or SEQ ID NO: 7.

Embodiment 18B: The composition of any one of embodiments 11B-17B, further comprising a secondary crosslinker linked to the repeat component by a linker.

Embodiment 19B: The composition of embodiment 18B, wherein the secondary crosslinker is a chemical crosslinker.

Embodiment 20B: The composition of embodiment 19B, wherein the secondary crosslinker comprises either a GB1A or GB1B

Embodiment 21B: The composition of embodiment 18B, wherein the secondary crosslinker comprises either a SpyCatcher or SpyTag.

Embodiment 22B: The composition of any one of embodiments 11B-20B, wherein the binding of biotin or a derivative thereof to the receptors of streptavidin modulates the mechanical properties of the biopolymer composition.

Embodiment 23B: A biopolymer composition comprising a plurality of polypeptide monomer composition according to any one of embodiments 11B-22B linked together.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of modulating mechanical properties of a biopolymer composition, the method comprising:
a) solubilizing a plurality of polypeptide monomers, each monomer comprising:
i) a first crosslinker; and
ii) a repeat component comprising a plurality of amino acid repeats linearly connected together linked to the first crosslinker; and
b) adding a concentration of biotin or a derivative thereof to the solubilized polypeptide monomers;
i) wherein solubilizing the polypeptide monomers allows for the first crosslinkers to associate together to form the biopolymer composition; and
ii) wherein the concentration of biotin or a derivative thereof modulates the mechanical properties of the biopolymer composition.

2. The method of claim 1, wherein the first crosslinker comprises a streptavidin polypeptide, an avidin polypeptide, or a derivative thereof.

3. The method of claim 1, wherein the repeat component comprises an ankyrin component comprising a plurality of ankyrin repeats linearly connected together linked to the first crosslinker.

4. The composition of claim 3, wherein the ankyrin component comprises 3 to 24 ankyrin repeats.

5. The method of claim 1, wherein the repeat component comprises SEQ ID NO: 6 or SEQ ID NO: 7.

6. The method of claim 1, wherein each monomer further comprises a secondary crosslinker linked to the repeat component by a linker.

7. The method of claim 6, wherein the secondary crosslinker is a chemical crosslinker.

8. The method of claim 7, wherein the secondary crosslinker comprises either a GB1A or GB1B or wherein the secondary crosslinker comprises either a SpyCatcher or SpyTag.

9. The method of claim 1, wherein the concentration of biotin or a derivative thereof is about 0.01 mM to 10 mM.

10. A polypeptide monomer composition effective as a nano spring, the composition comprising: a first crosslinker linked to repeat component comprising a plurality of amino acid repeats linearly connected.

11. The composition of claim 10 further comprising biotin or a derivative thereof bound to the receptor of the first crosslinker.

12. The composition of claim 10, wherein the first crosslinker comprises a streptavidin polypeptide, an avidin polypeptide, or a derivative thereof.

13. The composition of claim 10, wherein the repeat component comprises an ankyrin component comprising a plurality of ankyrin repeats linearly connected together linked to the first crosslinker.

14. The composition of claim 13, wherein the ankyrin component comprises 3 to 24 ankyrin repeats.

15. The composition of claim 10, wherein the repeat component comprises SEQ ID NO: 6 or SEQ ID NO: 7.

16. The composition of claim 10, further comprising a secondary crosslinker linked to the repeat component by a linker.

17. The composition of claim 16, wherein the secondary crosslinker is a chemical crosslinker.

18. The composition of claim 17, wherein the secondary crosslinker comprises either a GB1A or GB1B or wherein the secondary crosslinker comprises either a SpyCatcher or SpyTag.

19. The composition of claim 11, wherein the binding of biotin or a derivative thereof to the receptors of streptavidin modulates the mechanical properties of the biopolymer composition.

20. A biopolymer composition comprising a plurality of polypeptide monomer composition, according to claim 10, linked together.
